(19) United States
(12) Patent Application Publication
(10) Pub. No.: US 2025/0230493 A1
(43) Pub. Date: Jul. 17, 2025

(54) METHOD FOR COMBINED GENOME METHYLATION AND VARIATION ANALYSES

(71) Applicants: CZ Biohub SF, LLC, San Francisco, CA (US); The Board of Trustees of the Leland Stanford Junior University, Stanford, CA (US)

(72) Inventors: Charles Gawad, Stanford, CA (US); Veronica Gonzalez-Pena, Stanford, CA (US); David J. Klein, Los Altos, CA (US)

(21) Appl. No.: 18/706,875

(22) PCT Filed: Dec. 6, 2022
(86) PCT No.: PCT/US2022/051961

§ 371 Date: May 2, 2024

Related U.S. Application Data
(60) Provisional application No. 63/286,388, filed on Dec. 6, 2021.

(51) Int. Cl. C12Q 1/6869; C12N 15/10; C12Q 1/34; C12Q 1/6806

(52) U.S. Cl. CPC C12Q 1/6869; C12N 15/1068; C12Q 1/34; C12Q 1/6806; C12Q 2600/154; G01N 2333/922; G01N 2333/924; G01N 2333/978

(57) ABSTRACT

Provided herein is a method of identifying modified cytosines in genomic DNA in a biological sample. The method includes isolating, from the biological sample, nucleic acids comprising genomic DNA comprising cytosines and modified cytosines, contacting the isolated genomic DNA under conditions resulting in deamination of the genomic DNA thereby converting at least some of the cytosines in the genomic DNA to uracil and at least some of the modified cytosines to thymine, contacting the deaminated, isolated the genomic DNA with an enzyme to remove uracil from the genomic DNA, amplifying the genomic DNA lacking uracil using primary-directed template amplification, and sequencing the genomic DNA, wherein the sequencing identifies the modified cytosines in the genomic DNA of the single cell.

### CROSS-REFERENCE TO RELATED APPLICATION

This application claims priority to U.S. Provisional Patent Application No. 63/286,388, filed Dec. 6, 2021, which is incorporated by reference herein in its entirety.

### BACKGROUND

Single-cell transcriptomic and epigenomic profiling have identified new biological features of cells within tissues, enabling the discovery of new cellular states and processes that influence both healthy tissue homeostasis and aberrant disease-associated phenotypes. Single-cell genome sequencing has begun to characterize the diversity and evolution of cells within tissues, identifying the contributions of somatic mutations to the diseased states of both malignant and nonmalignant tissues. Still, the connection between altered cell states and underlying somatic genetic alterations has been difficult to study without tools that can accurately detect both nucleic acid changes in the same cells.

### BRIEF SUMMARY

Provided herein is a method of identifying modified cytosines in genomic DNA in a biological sample. The method includes isolating, from the biological sample, nucleic acids comprising genomic DNA comprising cytosines and modified cytosines, contacting the isolated genomic DNA under conditions resulting in deamination of the genomic DNA thereby converting at least some of the cytosines in the genomic DNA to uracil and at least some of the modified cytosines to thymine, contacting the deaminated, isolated the genomic DNA with an enzyme to remove uracil from the genomic DNA, amplifying the genomic DNA lacking uracil using primary-directed template amplification, and sequencing the genomic DNA, wherein the sequencing identifies the modified cytosines in the genomic DNA of the single cell.

### BRIEF DESCRIPTION OF THE DRAWINGS

FIG. 1 is a schematic showing the overview of an example experimental workflow of the herein provided method.

FIG. 2 is a graph showing the DNA yield with increasing incubation time.

FIG. 3 is a bar graph showing the fraction of reads aligning to the human genome over time.

FIG. 4 is a graph showing the predicted genome coverage with increasing incubation time.

FIG. 5 is a graph showing the coefficient of variation of genome coverage.

FIG. 6 is a graph showing the number of variants called over time with low-pass sequencing.

FIG. 7 is a graph showing the proportion of variants in each class over time.

FIG. 8 is a graph showing genome coverage with deep with deep sequencing after 30 minute incubation.

FIG. 9 is a bar graph showing coefficient of variation of variation of coverage with deep sequencing.

FIG. 10 is a bar graph showing estimated heterozygous single nucleotide polymorphism (SNP) sensitivity with deep sequencing.

FIG. 11 is a bar graph showing total number of SNPs detected with deep sequencing.

FIG. 12 is a bar graph showing the number of somatic variants detected with deep sequencing.

FIG. 13 is a bar graph showing the proportion of variants in each class with deep sequencing.

FIG. 14 is a graph showing MgCl₂ increases the yield from single-cell-Methyl-PTA. Single GM12878 cells sorted into LoBind PCR tubes containing 3 μL of BioSkryb cell buffer were lysed and heat-induced cytosine-deaminated at 60° C. for 4 hours in the presence of various concentrations of MgCl₂ followed by uracil removal with USER and PTA. The line graph indicates the median DNA yield from three independent scMethyl-PTA experiments, and the corresponding standard error.

FIG. 15 is a graph showing DTT increases the yield from single-cell-Methyl-PTA. Single GM12878 cells sorted into LoBind PCR tubes containing 3 μL of BioSkryb cell buffer were lysed and heat-induced cytosine-deaminated at 60° C. for 4 hours in the presence of various concentrations of DTT followed by uracil removal and PTA. The line graph indicates the median DNA yield from three independent scMethyl-PTA experiments, and the corresponding standard error.

FIG. 16 is a graph showing MgCl₂ and DTT additives have a synergistic effect on the yield from single-cell-Methyl-PTA. Single GM12878 cells sorted into LoBind PCR tubes containing 3 μL of BioSkryb cell buffer were lysed and heat-induced Cytosine-deaminated at 56° C. for 4 hours in the presence of 0.63 mM MgCl₂ and/or 25 mM DTT followed by uracil removal and PTA. The line graph indicates the median DNA yield from three independent scMethyl-PTA experiments, and the corresponding standard error.

FIG. 17 is a bar graph showing longer heat-induced deamination step of single-cell-Methyl-PTA results in an increased number of somatic mutations. Single GM12878 cells sorted into LoBind PCR tubes containing 3 μL of BioSkryb cell buffer were lysed and heat-induced Cytosine-deaminated at 56° C. for various amounts of time in the presence of 0.63 mM MgCl₂ and/or 25 mM DTT followed by uracil removal with USER enzyme and PTA. Sequencing libraries were prepared using Nextera Flex DNA library prep kit, and then underwent 30× whole genome sequencing followed by variant calling and whole genome somatic mutation number estimates using SCAN2. The graph indicates the calculated number of somatic mutations.

FIG. 18 are graphs showing longer heat-induced deamination step of single-cell-Methyl-PTA results in selective depletion of promoters and CpG islands. Single GM12878 cells sorted into LoBind PCR tubes containing 3 μL of BioSkryb cell buffer were lysed and heat-induced Cytosine-deaminated at 56° C. for various amounts of time in the presence of 0.63 mM MgCl₂ and/or 25 mM DTT followed by uracil removal with USER enzyme and PTA. Sequencing libraries were prepared using Nextera Flex DNA library prep kit, and then underwent 30× whole genome sequencing followed by alignment using Sentieon and calculation of the fraction of specific genomic regions covered using a bed files downloaded from the University of California Santa Cruz Table Browser with Mosdepth. FIG. 18 shows depletion of regions with high rates of cytosine including CpG Islands that have lower rates of 5-methyl-cytosine at CpG sites than other regions of the genome.

### DETAILED DESCRIPTION

There have been methods developed for detecting both genome variants and gene expression in the same cells, but these strategies have been limited by the suboptimal quality of the genome sequencing data. Single-cell methylation detection methods have also been developed, most of which rely on bisulfite conversion of unmethylated cytosine. However, these methods are also hampered by poor genome recovery due to nucleic acid destruction and genome loss during sample processing, which precludes concurrent detection of most genomic variants. Moreover, bisulfite-mediated methods rely on the absence of deamination events to identify 5mc sites, which requires a high conversion rate to distinguish 5mC from wild-type unconverted cytosine—this also makes somatic variant calling difficult due to the high background false positive rate. Approaches to optimize both bisulfite conversion and enrichment of genomic regulatory regions still suffer the requirement for high conversion rates to identify 5mC and also still suffers from limited genome coverage.

As described herein, the provided methods take advantage of the preferential deamination of 5mC over unmethylated cytosine under basic conditions and/or elevated temperature to create low-density cytosine deamination events that leave most of the remaining genome intact. As cytosine is converted to uracil while 5mC is converted to thymine, deaminated unmethylated cytosine (uracil) can be removed from that DNA strand using uracil DNA glycosylase. Digital inferences are then made of the methylation state of genome regulatory regions based on the enrichment of cytosine to thymine variants (representing a previous 5mC). Further, the coverage depth can be used as a proxy for uracil removal to corroborate those methylation calls, as well as estimate the allele frequency of heterozygous sites to infer the methylation status of one or both alleles. The result is a mostly intact genome that has 5mC marks, which can be used with a genome amplification method for concurrent variant and methylation detection in the same cell.

Primary Template-Directed Amplification (PTA), a whole genome amplification method that is able to call greater than 90% of small genomic variants and copy number alterations from the same cells as a result of the improved genome recovery and uniformity with a low error rate. As described herein, the combination of the provided 5mC marking strategy with PTA (methyl-PTA) to recover over 90% of the genome, enables subtraction-free, accurate genome-wide calling of small genomic, copy number, and other structural variants, as well as the methylation status of regulatory regions in the same single cells. This method can be utilized to better evaluate the state of early embryos, as well as uncover both genetic and epigenetic mechanisms of therapy resistance in acute myeloid leukemia samples from patients receiving both hypomethylating and chemotherapies.

Unless defined otherwise, all technical and scientific terms used herein have the same meaning as is commonly understood by one of ordinary skill in the art to which these inventions belong.

Throughout this disclosure, numerical features are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms“comprises” and/or“comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term“about” in reference to a number or range of numbers is understood to mean the stated number and numbers+/−10% thereof, or 10% below the lower listed limit and 10% above the higher listed limit for the values listed for a range.

The terms “subject” or “patient” or “individual”, as used herein, refer to animals, including mammals, such as, e.g., humans, veterinary animals (e.g., cats, dogs, cows, horses, sheep, pigs, etc.) and experimental animal models of diseases (e.g., mice, rats). In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook el al, 1989”); DNA Cloning: A practical Approach, Volumes I and II (D. N. Glover ed. 1985); Oligonucleotide Synthesis (MJ. Gait ed. 1984); Nucleic Acid Hybridization (B. D. Hames & S. J. Higgins eds. (1985; Transcription and Translation (B. D. Hames & S. J. Higgins, eds. (1984; Animal Cell Culture (R. I. Freshney, ed. (1986; Immobilized Cells and Enzymes (IRL Press, (1986; B. Perbal, A practical Guide To Molecular Cloning (1984); F. M. Ausubel et al. (eds.), Current Protocols in Molecular Biology, John Wiley & Sons, Inc. (1994); among others.

The term “nucleic acid” encompasses multi-stranded, as well as single-stranded molecules. In double- or triple-stranded nucleic acids, the nucleic acid strands need not be coextensive (i.e., a double-stranded nucleic acid need not be double-stranded along the entire length of both strands). Nucleic acid templates described herein may be any size depending on the sample (from small cell-free DNA fragments to entire genomes), including but not limited to 50-300 bases, 100-2000 bases, 100-750 bases, 170-500 bases, 100-5000 bases, 50-10,000 bases, or 50-2000 bases in length. In some instances, templates are at least 50, 100, 200, 500, 1000, 2000, 5000, 10,000, 20,000 50,000, 100,000, 200,000, 500,000, 1,000,000 or more than 1,000,000 bases in length. Methods described herein provide for the amplification of nucleic acid acids, such as nucleic acid templates. Methods described herein additionally provide for the generation of isolated and at least partially purified nucleic acids and libraries of nucleic acids. In some instances, methods described herein provide for extracted nucleic acids (e.g., extracted from tissues, cells, or media). Nucleic acids include but are not limited to those comprising DNA, RNA, circular RNA, mtDNA (mitochondrial DNA), cfDNA (cell free DNA), cfRNA (cell free RNA), siRNA (small interfering RNA), cffDNA (cell free fetal DNA), mRNA, tRNA, rRNA, miRNA (microRNA), synthetic polynucleotides, polynucleotide analogues, any other nucleic acid consistent with the specification, or any combinations thereof. The length of polynucleotides, when provided, are described as the number of bases and abbreviated, such as nt (nucleotides), bp (bases), kb (kilobases), or gb (gigabases).

As used herein, the term “unique molecular identifier (UMI)” refers to a unique nucleic acid sequence that is attached to each of a plurality of nucleic acid molecules. When incorporated into a nucleic acid molecule, an UMI in some instances is used to correct for subsequent amplification bias by directly counting UMIs that are sequenced after amplification. The design, incorporation and application of UMIs is described, for example, in Int. Pat. Appl. Pub. No. WO 2012/142213, Islam et al. Nat. Methods (2014) 11: 163-166, Kivioja, T. et al. Nat. Methods (2012) 9: 72-74, Brenner et al. (2000) PNAS 97(4), 1665, and Hollas and Schuler, (2003) Conference: 3rd International Workshop on Algorithms in Bioinformatics, Volume: 2812.

As used herein, the term “barcode” refers to a nucleic acid tag that can be used to identify a sample or source of the nucleic acid material. Thus, where nucleic acid samples are derived from multiple sources, the nucleic acids in each nucleic acid sample are in some instances tagged with different nucleic acid tags such that the source of the sample can be identified. Barcodes, also commonly referred to indexes, tags, and the like, are well known to those of skill in the art. Any suitable barcode or set of barcodes can be used. See, e.g., non-limiting examples provided in U.S. Pat. No. 8,053,192 and Int. Pat. Appl. Pub. No. WO2005/068656. Barcoding of single cells can be performed as described, for example, in U.S. Pat. Appl. Pub. No. 2013/0274117.

The terms “solid surface,” “solid support” and other grammatical equivalents herein refer to any material that is appropriate for or can be modified to be appropriate for the attachment of the primers, barcodes and sequences described herein. Exemplary substrates include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon, nitrocellulose, ceramics, resins, silica, silica-based materials (e.g., silicon or modified silicon), carbon, metals, inorganic glasses, plastics, optical fiber bundles, and a variety of other polymers. In some embodiments, the solid support comprises a patterned surface suitable for immobilization of primers, barcodes and sequences in an ordered pattern.

As used herein, the term “biological sample” includes, but is not limited to, tissues, cells, biological fluids and isolates thereof. Cells or other samples used in the methods described herein are in some instances isolated from human patients, animals, plants, soil or other samples comprising microbes such as bacteria, fungi, protozoa, etc. In some instances, the biological sample is of human origin. In some instances, the biological is of non-human origin. The cells in some instances undergo Primary Template-Directed Amplification (PTA) methods described herein and sequencing. Variants detected throughout the genome or at specific locations can be compared with all other cells isolated from that subject to trace the history of a cell lineage for research or diagnostic purposes. In some instances, variants are confirmed through additional methods of analysis such as direct PCR sequencing. The biological sample can be a low input sample. Low input samples include, but are not limited to, a forensic sample, ancient genomic fragments, and unculturable microbes. Optionally, the low input sample is a single cell. Cells can be of any type from any origin. For example, the single cell can be a primary cell. Optionally, the single cell originates from liver, skin, kidney, blood, or lung. Optionally, the single cell is a cancer cell, neuron, glial cell, or fetal cell. Single cells can be isolated by a variety of methods including, for example, flow cytometry.

Provided herein is a method of identifying modified cytosines in genomic DNA in a biological sample. The method includes isolating, from the biological sample, nucleic acids comprising genomic DNA comprising cytosines and modified cytosines, contacting the isolated genomic DNA under conditions resulting in deamination of the genomic DNA thereby converting at least some of the cytosines in the genomic DNA to uracil and at least some of the modified cytosines to thymine, contacting the deaminated, isolated the genomic DNA with an enzyme to remove uracil from the genomic DNA, amplifying the genomic DNA lacking uracil using primary-directed template amplification, and sequencing the genomic DNA library, wherein the sequencing identifies the modified cytosines in the genomic DNA of the single cell. In the provided methods, the sequencing can also include detecting variants in the genomic DNA.

As described herein, the modified cytosines can be 5′-methylcytosines or 5′-hydroxymethylcytosines. The identifying of the provided method can include identifying the methylated cytosines in genomic DNA. Optionally, the identifying comprises identifying CpG islands in the genomic DNA.

Deamination of the cytosines can include contacting the isolated genomic DNA with an enzyme that converts cytosine to uracil and 5′-methylcytosine to thymine. Optionally, the enzyme is a Apolipoprotein B mRNA Editing Catalytic Polypeptide-like (APOBEC) family protein. Optionally, the APOBEC family protein is APOBEC1 (A1), Activation Induced Deaminase (AID), APOBEC2 (A2), APOBEC3A-H (A3A-H), APOBEC4 (A4) proteins or a combination thereof. Optionally, the enzyme is AID.

Deamination can also include contacting the isolated genomic DNA with an enzyme converting cytosine to uracil and 5′-hydroxymethylcytosine to thymine. Enzymes that can be used to convert 5′-hydroxymethylcytosine to thymine include, but are not limited to, Single-Strand-Selective Monofunctional Uracil-DNA Glycosylase 1 (SMUG1).

Deamination can also be carried out by incubating the genomic DNA at a certain temperature. Thus, the provided methods can include a step of contacting the isolated genomic DNA that comprises incubating the genomic DNA at a certain temperature. The temperature can be between 50° C. and 100° C. The temperature can be between 50° C. and 75° C., 50° C. and 70° C., 50° C. and 65° C. or 50° C. and 60° C. Deamination can be carried out over a period of time, for example, between 30 and 240 minutes. Optionally, deamination is carried out over a period of time between four and 24 hours or betwwen four and 12 hours.

The provided methods can occur in the present of magnesium, dithiothreitol (DTT) or both. Optionally, the method is carried out in the presence of 0.6 to 20 mM magnesium, 0.6 to 15 mM magnesium, 0.6 to 10 mM magnesium or 0.6 to 5 mM magnesium. The magnesium can be in the form of MgCL₂. The methods can be carried out in the presence of DTT. For example, the methods can be carried out in the presence of 5 to 100 mM DTT, 5 to 75 mM DTT, 5 to 50 mM DTT or 5 to 25 mM DTT. The methods can be carried out in the presence of both magnesium and DTT. For example, the methods can be carried out in the presence of 0.6 to 20 mM magnesium, 0.6 to 15 mM magnesium, 0.6 to 10 mM magnesium or 0.6 to 5 mM magnesium and 5 to 100 mM DTT, 5 to 75 mM DTT, 5 to 50 mM DTT or 5 to 25 mM DTT. Thus, the methods can be carried out in the presence of 0.6 to 10 mM magnesium and 5 to 100, 5 to 75, 5 to 50 or 5 to 25 mM DTT.

The amounts of magnesium and DTT used in the provided methods can be used in any amounts as described herein in any combination with the temperatures and times provided herein. In some instances, the method is carried out in the presence of 5 to 100 mM DTT, 0.6 to 20 mM magnesium and deamination occurs at a temperature between 50° C. to 75° C. for 4 to 24 hours. In some instances, the method is carried out in the presence of 5 to 100 mM DTT, 0.6 to 20 mM magnesium and deamination occurs at a temperature between 50° C. to 65° C. for 4 to 24 hours. In some instances, the method is carried out in the presence of 5 to 75 mM DTT, 0.6 to 20 mM magnesium and deamination occurs at a temperature between 50° C. to 65° C. for 4 to 24 hours. In some instances, the method is carried out in the presence of 5 to 75 mM DTT, 0.6 to 20 mM magnesium and deamination occurs at a temperature between 50° C. to 65° C. for 4 to 12 hours. In some instances, the method is carried out in the presence of 5 to 75 mM DTT, 0.6 to 10 mM magnesium and deamination occurs at a temperature between 50° C. to 65° C. for 4 to 12 hours. In some instances, the method is carried out in the presence of 5 to 50 mM DTT, 0.6 to 10 mM magnesium and deamination occurs at a temperature between 50° C. to 65° C. for 4 to 24 hours. In some instances, the method is carried out in the presence of 5 to 50 mM DTT, 0.6 to 10 mM magnesium and deamination occurs at a temperature between 50° C. to 65° C. for 4 to 12 hours. In some instances, the method is carried out in the presence of 5 to 25 mM DTT, 0.6 to 10 mM magnesium and deamination occurs at a temperature between 50° C. to 60° C. for 4 to 12 hours.

Optionally, isolating the nucleic acids can include contacting the biological sample with a lysis buffer or liquid with an alkaline pH. The lysis buffer or liquid can have a pH between 9 to 14.

In the provided methods, contacting the deaminated, isolated genomic DNA can include contacting with an enzymatic buffer comprising the enzyme. The enzyme can be uracil DNA glycosylase. Optionally, the enzymatic buffer further comprises DNA glycosylase-lyase Endonuclease VIII to remove the abasic sites created by an enzyme like uracil DNA glycosylase.

In the provided methods, the uracil removal steps can occur over a period of time. For example, the period of time is between 15 to 60 minutes, e.g., 30 minutes.

In the provided methods, the uracil removal steps can include contacting at a certain temperature. For example, contacting the deaminated genomic DNA can include incubating the genomic DNA at a temperature from 30° C. to 45° C.

In the provided methods, a percentage of cytosines and modified cytosines are converted. By way of example, 0.1% to 10% of the cytosines can be converted to uracil. Optionally, 1% to 10% of the cytosines can be converted to uracil. Optionally, 5% to 10% of the cytosines are converted to uracil. If the modified cytosines are 5′-methylcytosines, the provided methods can convert from 0.1% to 10% to thymine. Optionally, 5% to 10% of the 5′-methylcytosines are converted to thymine. Optionally, 10% to 100% of the cytosines are converted to uracil. Optionally, 10% to 100% of the 5′-methylcytosines are converted to thymine. If the modified cytosines are 5′-hydroxymethylcytosines, the provided methods can convert from 0.1% to 10% to thymine. Optionally, 1% to 10% of the 5′-hydroxymethylcytosines are converted to thymine. Optionally, 5% to 10% of the 5′-hydroxymethylcytosines are converted to thymine. Optionally, 10% to 100% of the cytosines are converted to uracil. Optionally, 10% to 100% of the 5′-methylcytosines are converted to thymine.

The herein provided methods can also include analyzing nucleic acids other than genomic DNA. Thus, in the provided methods the nucleic acids can also include mRNAs and the method can include the additional steps of converting the mRNAs in the nucleic acid sample to complementary DNAs (cDNAs) using reverse transcription and template switching oligonucleotides, and amplifying and sequencing the cDNAs. Optionally, the mRNAs comprise polyadenylated mRNAs. Optionally, at least some of the polynucleotides of the cDNA library includes a barcode. The barcode can include a cell barcode or a sample barcode. Optionally, the mRNA transcripts are amplified via template-switching reverse transcription.

The primary-directed template amplification can include contacting the genomic DNA with at least one amplification primer, at least one nucleic acid polymerase, and a mixture of nucleotides, wherein the mixture of nucleotides comprises at least one terminator nucleotide which terminates nucleic acid replication by the polymerase, amplifying at least some of the genomic DNA to generate a plurality of terminated amplification products, wherein the replication proceeds by strand displacement replication, and ligating the molecules obtained in step (ii) to adaptors, thereby generating a genomic DNA library. Optionally, the method further includes removing at least one terminator nucleotide from the terminated amplification products. The plurality of terminated amplification products can include an average of 1000-2000 bases in length. Optionally, the plurality of terminated amplification products are 250-1500 bases in length. Optionally, at least some of the amplification products comprise a cell barcode or a sample barcode.

The cDNA library can include any number of genes. For example, the cDNA library can include at least 10,000 genes.

The cDNA and genomic DNA can be pooled prior to sequencing. Thus, the method can include pooling the cDNA library and the genomic DNA library prior to sequencing.

The methods described herein may require isolation of single cells for analysis. Any method of single cell isolation may be used with PTA, such as mouth pipetting, micro pipetting, flow cytometry/FACS, microfluidics, methods of sorting nuclei (tetraploid or other), or manual dilution. Such methods are aided by additional reagents and steps, for example, antibody-based enrichment (e.g., circulating tumor cells), other small-molecule or protein-based enrichment methods, or fluorescent labeling. In some instances, a method of multiomic analysis described herein comprises mechanical or enzymatic dissociate of cells from larger tissues.

The data obtained from single-cell analysis methods utilizing PTA described herein may be compiled into a database. Described herein are methods and systems of bioinformatic data integration. Data from the proteome, genome, transcriptome, methylome or other data is in some instances combined/integrated into a database and analyzed. Bioinformatic data integration methods and systems in some instances comprise one or more of protein detection (FACS and/or NGS), mRNA detection, and/or genome variance detection. In some instances, this data is correlated with a disease state or condition. In some instances, data from a plurality of single cells is compiled to describe properties of a larger cell population, such as cells from a specific sample, region, organism, or tissue. In some instances, protein data is acquired from fluorescently labeled antibodies which selectively bind to proteins on a cell. In some instances, a method of protein detection comprises grouping cells based on fluorescent markers and reporting sample location post-sorting. In some instances, a method of protein detection comprises detecting sample barcodes, detecting protein barcodes, comparing to designed sequences, and grouping cells based on barcode and copy number. In some instances, protein data is acquired from barcoded antibodies which selectively bind to proteins on a cell. In some instances, transcriptome data is acquired from sample and RNA specific barcodes. In some instances, a method of mRNA detection comprises detecting sample and RNA specific barcodes, aligning to genome, aligning to RefSeq/Encode, reporting Exon/Intro/Intergenic sequences, analyzing exon-exon junctions, grouping cells based on barcode and expression variance and clustering analysis of variance and top variable genes. In some instances, genomic data is acquired from sample and DNA specific barcodes. In some instances, a method of genome variance detection comprises detecting sample and DNA specific barcodes, aligning to the genome, determine genome recovery and SNV mapping rate, filtering reads on exon-exon junctions, generating variant call file (VCF), and clustering analysis of variance and top variable mutations.

In some instances, the methods (e.g., multiomic PTA) described herein result in higher detection sensitivity and/or lower rates of false positives for the detection of mutations. In some instances a mutation is a difference between an analyzed sequence (e.g., using the methods described herein) and a reference sequence. Reference sequences are in some instances obtained from other organisms, other individuals of the same or similar species, populations of organisms, or other areas of the same genome. In some instances, mutations are identified on a plasmid or chromosome. In some instances, a mutation is an SNV (single nucleotide variation), SNP (single nucleotide polymorphism), or chromosomal structural variation including CNV (copy number variation, or CNA/copy number aberration). In some instances, a mutation is base substitution, insertion, or deletion. In some instances, a mutation is a transition, transversion, nonsense mutation, silent mutation, synonymous or non-synonymous mutation, non-pathogenic mutation, missense mutation, or frameshift mutation (deletion or insertion). In some instances, PTA results in higher detection sensitivity and/or lower rates of false positives for the detection of mutations when compared to methods such as in-silico prediction, ChIP-seq, GUIDE-seq, circle-seq, HTGTS (High-Throughput Genome-Wide Translocation Sequencing), IDLV (integration-deficient lentivirus), Digenome-seq, FISH (fluorescence in situ hybridization), or DISCOVER-seq.

Described herein are nucleic acid amplification methods, such as “Primary Template-Directed Amplification (PTA).” PTA is described in and demonstrated in WO 2021/022085, which is incorporated by reference herein in its entirety. In some instances, PTA is combined with other analysis workflows for multiomic analysis. With the PTA method, amplicons are preferentially generated from the primary template (“direct copies”) using a polymerase (e.g., a strand displacing polymerase). Consequently, errors are propagated at a lower rate from daughter amplicons during subsequent amplifications compared to MDA. The result is an easily executed method that, unlike existing WGA protocols, can amplify low DNA input including the genomes of single cells with high coverage breadth and uniformity in an accurate and reproducible manner. Moreover, the terminated amplification products can undergo direction ligation after removal of the terminators, allowing for the attachment of a cell barcode to the amplification primers so that products from all cells can be pooled after undergoing parallel amplification reactions. In some instances, template nucleic acids are not bound to a solid support. In some instances, direct copies of template nucleic acids are not bound to a solid support. In some instances, one or more primers are not bound to a solid support. In some instances, no primers are not bound to a solid support. In some instances, a primer is attached to a first solid support, and a template nucleic acid is attached to a second solid support, wherein the first and the second solid supports are not the same. In some instances, PTA is used to analyze single cells from a larger population of cells. In some instances, PTA is used to analyze more than one cell from a larger population of cells, or an entire population of cells.

Primers and/or template switching oligonucleotides can also be affixed to solid substrate to facilitate reverse transcription and template switching of the mRNA polynucleotides. In this arrangement a portion of the RT or template switching reaction occurs in the bulk solution of the device, where the second step of the reaction occurs in proximity to the surface. In other arrangements the primer of template switch oligonucleotide is allowed to be released from the solid substrate to allow the entire reaction to occur above the surface in the solution. In a polyomic approach the primers for the multistage reaction in some instances is affixed to the solid substrate or combined with beads to accomplish combinations of multistage primers.

Described herein are methods employing nucleic acid polymerases with strand displacement activity for amplification. In some instances, such polymerases comprise strand displacement activity and low error rate. In some instances, such polymerases comprise strand displacement activity and proofreading exonuclease activity, such as 3′->5′ proofreading activity. In some instances, nucleic acid polymerases are used in conjunction with other components such as reversible or irreversible terminators, or additional strand displacement factors. In some instances, the polymerase has strand displacement activity, but does not have exonuclease proofreading activity. For example, in some instances such polymerases include bacteriophage phi29 (F29) polymerase, which also has very low error rate that is the result of the 3′->5′ proofreading exonuclease activity (see, e.g., U.S. Pat. Nos. 5,198,543 and 5,001,050). In some instances, non-limiting examples of strand displacing nucleic acid polymerases include, e.g., genetically modified phi29 (F29) DNA polymerase, Klenow Fragment of DNA polymerase I (Jacobsen et al., Eur. J. Biochem. 45:623-627 (1974)), phage M2 DNA polymerase (Matsumoto et al., Gene 84:247 (1989)), phage phiPRDI DNA polymerase (Jung et al., Proc. Natl. Acad. Sci. USA 84:8287 (1987); Zhu and Ito, Biochim. Biophys. Acta. 1219:267-276 (1994)), Bst DNA polymerase (e.g., Bst large fragment DNA polymerase (Exo(−) Bst; Aliotta et al., Genet. Anal. (Netherlands) 12: 185-195 (1996)), exo(−)Bca DNA polymerase (Walker and Linn, Clinical Chemistry 42: 1604-1608 (1996)), Bsu DNA polymerase, VentRDNA polymerase including VentR (exo-) DNA polymerase (Kong et al., J. Biol. Chem. 268: 1965-1975 (1993)), Deep Vent DNA polymerase including Deep Vent (exo-) DNA polymerase, IsoPol DNA polymerase, DNA polymerase I, Therminator DNA polymerase, T5 DNA polymerase (Chatteijee et al., Gene 97: 13-19 (1991)), Sequenase (U.S. Biochemicals), T7 DNA polymerase, T7-Sequenase, T7 gp5 DNA polymerase, PRDI DNA polymerase, T4 DNA polymerase (Kaboord and Benkovic, Curr. Biol. 5: 149-157 (1995)). Additional strand displacing nucleic acid polymerases are also compatible with the methods described herein. The ability of a given polymerase to carry out strand displacement replication can be determined, for example, by using the polymerase in a strand displacement replication assay (e.g., as disclosed in U.S. Pat. No. 6,977,148). Such assays in some instances are performed at a temperature suitable for optimal activity for the enzyme being used, for example, 32° C. for phi29 DNA polymerase, from 46° C. to 64° C. for exo(−) Bst DNA polymerase, or from about 60° C. to 70° C. for an enzyme from a hyperthermophylic organism. Another useful assay for selecting a polymerase is the primer-block assay described in Kong et al., J. Biol. Chem. 268:1965-1975 (1993). The assay consists of a primer extension assay using an M1 3 ssDNA template in the presence or absence of an oligonucleotide that is hybridized upstream of the extending primer to block its progress. Other enzymes capable of displacement the blocking primer in this assay are in some instances useful for the disclosed method. In some instances, polymerases incorporate dNTPs and terminators at approximately equal rates. In some instances, the ratio of rates of incorporation for dNTPs and terminators for a polymerase described herein are about 1:1, about 1.5:1, about 2:1, about 3:1 about 4:1 about 5:1, about 10:1, about 20:1 about 50:1, about 100:1, about 200:1, about 500:1, or about 1000:1. In some instances, the ratio of rates of incorporation for dNTPs and terminators for a polymerase described herein are 1:1 to 1000:1, 2:1 to 500:1, 5:1 to 100:1, 10:1 to 1000:1, 100:1 to 1000:1, 500:1 to 2000:1, 50:1 to 1500:1, or 25:1 to 1000:1.

Described herein are methods of amplification wherein strand displacement can be facilitated through the use of a strand displacement factor, such as, e.g., helicase. Such factors are in some instances used in conjunction with additional amplification components, such as polymerases, terminators, or other component. In some instances, a strand displacement factor is used with a polymerase that does not have strand displacement activity. In some instances, a strand displacement factor is used with a polymerase having strand displacement activity. Without being bound by theory, strand displacement factors may increase the rate that smaller, double stranded amplicons are reprimed. In some instances, any DNA polymerase that can perform strand displacement replication in the presence of a strand displacement factor is suitable for use in the PTA method, even if the DNA polymerase does not perform strand displacement replication in the absence of such a factor. Strand displacement factors useful in strand displacement replication in some instances include (but are not limited to) BMRF1 polymerase accessory subunit (Tsurumi et al., J. Virology 67(12):7648-7653 (1993)), adenovirus DNA-binding protein (Zijderveld and van der Vliet, J. Virology 68(2): 1158-1164 (1994)), herpes simplex viral protein ICP8 (Boehmer and Lehman, J. Virology 67(2):711-715 (1993); Skaliter and Lehman, Proc. Natl. Acad. Sci. USA 91(22): 10665-10669 (1994)); single-stranded DNA binding proteins (SSB; Rigler and Romano, J. Biol. Chem. 270:8910-8919 (1995)); phage T4 gene 32 protein (Villemain and Giedroc, Biochemistry 35: 14395-14404 (1996); T7 helicase-primase; T7 gp2.5 SSB protein; Tte-UvrD (from Thermoanaerobacter tengcongensis), calf thymus helicase (Siegel et al., J. Biol. Chem. 267: 13629-13635 (1992)); bacterial SSB (e.g., E. coli SSB), Replication Protein A (RPA) in eukaryotes, human mitochondrial SSB (mtSSB), and recombinases, (e.g., Recombinase A (RecA) family proteins, T4 UvsX, T4 UvsY, Sak4 of Phage HK620, Rad51, Dmcl, or Radb). Combinations of factors that facilitate strand displacement and priming are also consistent with the methods described herein. For example, a helicase is used in conjunction with a polymerase. In some instances, the PTA method comprises use of a single-strand DNA binding protein (SSB, T4 gp32, or other single stranded DNA binding protein), a helicase, and a polymerase (e.g., SauDNA polymerase, Bsu polymerase, Bst2.0, GspM, GspM2.0, GspSSD, or other suitable polymerase). In some instances, reverse transcriptases are used in conjunction with the strand displacement factors described herein. In some instances, reverse transcriptases are used in conjunction with the strand displacement factors described herein. In some instances, amplification is conducted using a polymerase and a nicking enzyme (e.g., “NEAR”), such as those described in U.S. Pat. No. 9,617,586. In some instances, the nicking enzyme is Nt.BspQI, Nb.BbvCi, Nb.BsmI, Nb.BsrDI, Nb.BtsI, Nt.AlwI, Nt.BbvCI, Nt.BstNBI, Nt.CviPII, Nb.BpulOI, or Nt.BpulOI.

Described herein are amplification methods comprising use of terminator nucleotides, polymerases, and additional factors or conditions. For example, such factors are used in some instances to fragment the nucleic acid template(s) or amplicons during amplification. In some instances, such factors comprise endonucleases. In some instances, factors comprise transposases.

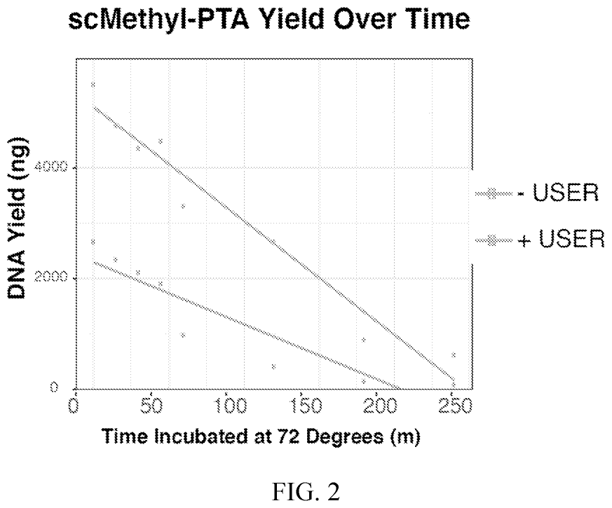

In some instances, mechanical shearing is used to fragment nucleic acids during amplification. In some instances, nucleotides are added during amplification that may be fragmented through the addition of additional proteins or conditions. For example, uracil is incorporated into amplicons; treatment with uracil D-glycosylase fragments nucleic acids at uracil-containing positions. Additional systems for selective nucleic acid fragmentation are also in some instances employed, for example an engineered DNA glycosylase that cleaves modified cytosine-pyrene base pairs. (Kwon, et al. Chem Biol. 2003, 10(4), 351) Described herein are amplification methods comprising use of terminator nucleotides, which terminate nucleic acid replication thus decreasing the size of the amplification products. Such terminators are in some instances used in conjunction with polymerases, strand displacement factors, or other amplification components described herein. In some instances, terminator nucleotides reduce or lower the efficiency of nucleic acid replication. Such terminators in some instances reduce extension rates by at least 99.9%, 99%, 98%, 95%, 90%, 85%, 80%, 75%, 70%, or at least 65%. Such terminators in some instances reduce extension rates by 50%-90%, 60%-80%, 65%-90%, 70%-85%, 60%-90%, 70%-99%, 80%-99%, or 50%-80%. In some instances terminators reduce the average amplicon product length by at least 99.9%, 99%, 98%, 95%, 90%, 85%, 80%, 75%, 70%, or at least 65%. Terminators in some instances reduce the average amplicon length by 50%-90%, 60%-80%, 65%-90%, 70%-85%, 60%-90%, 70%-99%, 80%-99%, or 50%-80%. In some instances, amplicons comprising terminator nucleotides form loops or hairpins which reduce a polymerase's ability to use such amplicons as templates. Use of terminators in some instances slows the rate of amplification at initial amplification sites through the incorporation of terminator nucleotides (e.g., dideoxynucleotides that have been modified to make them exonuclease-resistant to terminate DNA extension), resulting in smaller amplification products. By producing smaller amplification products than the currently used methods (e.g., average length of 50-2500 nucleotides in length for PTA methods as compared to an average product length of >10,000 nucleotides for MDA methods) PTA amplification products in some instances undergo direct ligation of adapters without the need for fragmentation, allowing for efficient incorporation of cell barcodes and unique molecular identifiers (UMI) (see FIG. 2A).

Terminator nucleotides are present at various concentrations depending on factors such as polymerase, template, or other factors. For example, the amount of terminator nucleotides in some instances is expressed as a ratio of non-terminator nucleotides to terminator nucleotides in a method described herein. Such concentrations in some instances allow control of amplicon lengths. In some instances, the ratio of terminator to non-terminator nucleotides is modified for the amount of template present or the size of the template. In some instances, the ratio of ratio of terminator to non-terminator nucleotides is reduced for smaller samples sizes (e.g., femtogram to picogram range). In some instances, the ratio of non-terminator to terminator nucleotides is about 2:1, 5:1, 7:1, 10:1, 20:1, 50:1, 100:1, 200:1, 500:1, 1000:1, 2000:1, or 5000:1. In some instances the ratio of non-terminator to terminator nucleotides is 2: 1-10:1, 5:1-20:1, 10:1-100:1, 20:1-200:1, 50:1-1000:1, 50:1-500:1, 75:1-150:1, or 100: 1-500:1. In some instances, at least one of the nucleotides present during amplification using a method described herein is a terminator nucleotide. Each terminator need not be present at approximately the same concentration; in some instances, ratios of each terminator present in a method described herein are optimized for a particular set of reaction conditions, sample type, or polymerase. Without being bound by theory, each terminator may possess a different efficiency for incorporation into the growing polynucleotide chain of an amplicon, in response to pairing with the corresponding nucleotide on the template strand. For example, in some instances a terminator pairing with cytosine is present at about 3%, 5%, 10%, 15%, 20%, 25%, or 50% higher concentration than the average terminator concentration. In some instances a terminator pairing with thymine is present at about 3%, 5%, 10%, 15%, 20%, 25%, or 50% higher concentration than the average terminator concentration. In some instances a terminator pairing with guanine is present at about 3%, 5%, 10%, 15%, 20%, 25%, or 50% higher concentration than the average terminator concentration. In some instances a terminator pairing with adenine is present at about 3%, 5%, 10%, 15%, 20%, 25%, or 50% higher concentration than the average terminator concentration. In some instances a terminator pairing with uracil is present at about 3%, 5%, 10%, 15%, 20%, 25%, or 50% higher concentration than the average terminator concentration. Any nucleotide capable of terminating nucleic acid extension by a nucleic acid polymerase in some instances is used as a terminator nucleotide in the methods described herein. In some instances, a reversible terminator is used to terminate nucleic acid replication. In some instances, a non-reversible terminator is used to terminate nucleic acid replication. In some instances, non-limited examples of terminators include reversible and non-reversible nucleic acids and nucleic acid analogs, such as, e.g., 3′ blocked reversible terminator comprising nucleotides, 3′ unblocked reversible terminator comprising nucleotides, terminators comprising T modifications of deoxynucleotides, terminators comprising modifications to the nitrogenous base of deoxynucleotides, or any combination thereof. In one embodiment, terminator nucleotides are dideoxynucleotides. Other nucleotide modifications that terminate nucleic acid replication and may be suitable for practicing the invention include, without limitation, any modifications of the r group of the 3′ carbon of the deoxyribose such as inverted dideoxynucleotides, 3′ biotinylated nucleotides, 3′ amino nucleotides, 3′-phosphorylated nucleotides, 3′-O-methyl nucleotides, 3′ carbon spacer nucleotides including 3′ C3 spacer nucleotides, 3′ Cl 8 nucleotides, 3′ Hexanediol spacer nucleotides, acyclonucleotides, and combinations thereof. In some instances, terminators are polynucleotides comprising 1, 2, 3, 4, or more bases in length. In some instances, terminators do not comprise a detectable moiety or tag (e.g., mass tag, fluorescent tag, dye, radioactive atom, or other detectable moiety). In some instances, terminators do not comprise a chemical moiety allowing for attachment of a detectable moiety or tag (e.g., “click” azide/alkyne, conjugate addition partner, or other chemical handle for attachment of a tag). In some instances, all terminator nucleotides comprise the same modification that reduces amplification to at region (e.g., the sugar moiety, base moiety, or phosphate moiety) of the nucleotide. In some instances, at least one terminator has a different modification that reduces amplification. In some instances, all terminators have a substantially similar fluorescent excitation or emission wavelengths. In some instances, terminators without modification to the phosphate group are used with polymerases that do not have exonuclease proofreading activity. Terminators, when used with polymerases which have 3′->5′ proofreading exonuclease activity (such as, e.g., phi29) that can remove the terminator nucleotide, are in some instances further modified to make them exonuclease-resistant. For example, dideoxynucleotides are modified with an alpha-thio group that creates a phosphorothioate linkage which makes these nucleotides resistant to the 3′->5′ proofreading exonuclease activity of nucleic acid polymerases. Such modifications in some instances reduce the exonuclease proofreading activity of polymerases by at least 99.5%, 99%, 98%, 95%, 90%, or at least 85%. Non-limiting examples of other terminator nucleotide modifications providing resistance to the 3′->5′ exonuclease activity include in some instances: nucleotides with modification to the alpha group, such as alpha-thio dideoxynucleotides creating a phosphorothioate bond, C3 spacer nucleotides, locked nucleic acids (LNA), inverted nucleic acids, 2′ Fluoro bases, 3′ phosphorylation, 2′-O-Methyl modifications (or other 2′-O-alkyl modification), propyne-modified bases (e.g., deoxycytosine, deoxyuridine), L-DNA nucleotides, L-RNA nucleotides, nucleotides with inverted linkages (e.g., 5′-5′ or 3′-3′), 5′ inverted bases (e.g., 5′ inverted 2′,3′-dideoxy dT), methylphosphonate backbones, and trans nucleic acids. In some instances, nucleotides with modification include base-modified nucleic acids comprising free 3′ OH groups (e.g., 2-nitrobenzyl alkylated HOMedU triphosphates, bases comprising modification with large chemical groups, such as solid supports or other large moiety). In some instances, a polymerase with strand displacement activity but without 3′->5′ exonuclease proofreading activity is used with terminator nucleotides with or without modifications to make them exonuclease resistant. Such nucleic acid polymerases include, without limitation, Bst DNA polymerase, Bsu DNA polymerase, Deep Vent (exo-) DNA polymerase, Klenow Fragment (exo-) DNA polymerase, Therminator DNA polymerase, and VentR (exo-).

Described herein are amplicon libraries resulting from amplification of at least one target nucleic acid molecule. Such libraries are in some instances generated using the methods described herein, such as those using terminators. Such methods comprise use of strand displacement polymerases or factors, terminator nucleotides (reversible or irreversible), or other features and embodiments described herein. In some instances, amplicon libraries generated by use of terminators described herein are further amplified in a subsequent amplification reaction (e.g., PCR). In some instances, subsequent amplification reactions do not comprise terminators. In some instances, amplicon libraries comprise polynucleotides, wherein at least 50%, 60%, 70%, 80%, 90%, 95%, or at least 98% of the polynucleotides comprise at least one terminator nucleotide. In some instances, the amplicon library comprises the target nucleic acid molecule from which the amplicon library was derived. The amplicon library comprises a plurality of polynucleotides, wherein at least some of the polynucleotides are direct copies (e.g., replicated directly from a target nucleic acid molecule, such as genomic DNA, RNA, or other target nucleic acid). For example, at least 5%, 10%, 20%, 30%, 40%, 50%, 60%, 70%, 80%, 90%, 95% or more than 95% of the amplicon polynucleotides are direct copies of the at least one target nucleic acid molecule. In some instances, at least 5% of the amplicon polynucleotides are direct copies of the at least one target nucleic acid molecule. In some instances, at least 10% of the amplicon polynucleotides are direct copies of the at least one target nucleic acid molecule. In some instances, at least 15% of the amplicon polynucleotides are direct copies of the at least one target nucleic acid molecule. In some instances, at least 20% of the amplicon polynucleotides are direct copies of the at least one target nucleic acid molecule. In some instances, at least 50% of the amplicon polynucleotides are direct copies of the at least one target nucleic acid molecule. In some instances, 3%-5%, 3-10%, 5%-10%, 10%-20%, 20%-30%, 30%-40%, 5%-30%, 10%-50%, or 15%-75% of the amplicon polynucleotides are direct copies of the at least one target nucleic acid molecule. In some instances, at least some of the polynucleotides are direct copies of the target nucleic acid molecule, or daughter (a first copy of the target nucleic acid) progeny. For example, at least 5%, 10%, 20%, 30%, 40%, 50%, 60%, 70%, 80%, 90%, 95% or more than 95% of the amplicon polynucleotides are direct copies of the at least one target nucleic acid molecule or daughter progeny. In some instances, at least 5% of the amplicon polynucleotides are direct copies of the at least one target nucleic acid molecule or daughter progeny. In some instances, at least 10% of the amplicon polynucleotides are direct copies of the at least one target nucleic acid molecule or daughter progeny. In some instances, at least 20% of the amplicon polynucleotides are direct copies of the at least one target nucleic acid molecule or daughter progeny. In some instances, at least 30% of the amplicon polynucleotides are direct copies of the at least one target nucleic acid molecule or daughter progeny. In some instances, 3%-5%, 3%-10%, 5%-10%, 10%-20%, 20%-30%, 30%-40%, 5%-30%, 10%-50%, or 15%-75% of the amplicon polynucleotides are direct copies of the at least one target nucleic acid molecule or daughter progeny. In some instances, direct copies of the target nucleic acid are 50-2500, 75-2000, 50-2000, 25-1000, 50-1000, 500-2000, or 50-2000 bases in length. In some instances, daughter progeny are 1000-5000, 2000-5000, 1000-10,000, 2000-5000, 1500-5000, 3000-7000, or 2000-7000 bases in length. In some instances, the average length of PTA amplification products is 25-3000 nucleotides in length, 50-2500, 75-2000, 50-2000, 25-1000, 50-1000, 500-2000, or 50-2000 bases in length. In some instance, amplicons generated from PTA are no more than 5000, 4000, 3000, 2000, 1700, 1500, 1200, 1000, 700, 500, or no more than 300 bases in length. In some instance, amplicons generated from PTA are 1000-5000, 1000-3000, 200-2000, 200-4000, 500-2000, 750-2500, or 1000-2000 bases in length. Amplicon libraries generated using the methods described herein in some instances comprise at least 1000, 2000, 5000, 10,000, 100,000, 200,000, 500,000 or more than 500,000 amplicons comprising unique sequences. In some instances, the library comprises at least 100, 200, 300, 400, 500, 600, 700, 800, 900, 1000, 1100, 1200, 1300, 1400, 1500, 2000, 2500, 3000, or at least 3500 amplicons. In some instances, at least 5%, 10%, 15%, 20%, 25%, 30% or more than 30% of amplicon polynucleotides having a length of less than 1000 bases are direct copies of the at least one target nucleic acid molecule. In some instances, at least 5%, 10%, 15%, 20%, 25%, 30% or more than 30% of amplicon polynucleotides having a length of no more than 2000 bases are direct copies of the at least one target nucleic acid molecule. In some instances, at least 5%, 10%, 15%, 20%, 25%, 30% or more than 30% of amplicon polynucleotides having a length of 3000-5000 bases are direct copies of the at least one target nucleic acid molecule. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are no more than 700-1200 bases in length.

In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1. In some instances, the ratio of direct copy amplicons and daughter amplicons to target nucleic acid molecules is at least 10:1, 100:1, 1000:1, 10,000:1, 100,000:1, 1,000,000:1, 10,000,000:1, or more than 10,000,000:1, wherein the direct copy amplicons are 700-1200 bases in length, and the daughter amplicons are 2500-6000 bases in length. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule. In some instances, the library comprises about 50-10,000, about 50-5,000, about 50-2500, about 50-1000, about 150-2000, about 250-3000, about 50-2000, about 500-2000, or about 500-1500 amplicons which are direct copies of the target nucleic acid molecule or daughter amplicons. The number of direct copies may be controlled in some instances by the number of PCR amplification cycles. In some instances, no more than 30, 25, 20, 15, 13, 11, 10, 9, 8, 7, 6, 5, 4, or 3 PCR cycles are used to generate copies of the target nucleic acid molecule. In some instances, about 30, 25, 20, 15, 13, 11, 10, 9, 8, 7, 6, 5, 4, or about 3 PCR cycles are used to generate copies of the target nucleic acid molecule. In some instances, 3, 4, 5, 6, 7, or 8 PCR cycles are used to generate copies of the target nucleic acid molecule. In some instances, 2-4, 2-5, 2-7, 2-8, 2-10, 2-15, 3-5, 3-10, 3-15, 4-10, 4-15, 5-10 or 5-15 PCR cycles are used to generate copies of the target nucleic acid molecule. Amplicon libraries generated using the methods described herein are in some instances subjected to additional steps, such as adapter ligation and further PCR amplification. In some instances, such additional steps precede a sequencing step.

Methods described herein may additionally comprise one or more enrichment or purification steps. In some instances, one or more polynucleotides (such as cDNA, PTA amplicons, or other polynucleotide) are enriched during a method described herein. In some instances, polynucleotide probes are used to capture one or more polynucleotides. In some instances, probes are configured to capture one or more genomic exons. In some instances, a library of probes comprises at least 1000, 2000, 5000, 10,000, 50,000, 100,000, 200,000, 500,000, or more than 1 million different sequences. In some instances, a library of probes comprises sequences capable of binding to at least 10, 20, 50, 100, 200, 500, 1000, 2000, 5000, 10,000 or more than 10,000 genes. In some instances, probes comprise a moiety for capture by a solid support, such as biotin. In some instances, an enrichment step occurs after a PTA step. In some instances, an enrichment step occurs before a PTA step. In some instances, probes are configured to bind genomic DNA libraries. In some instances, probes are configured to bind cDNA libraries.

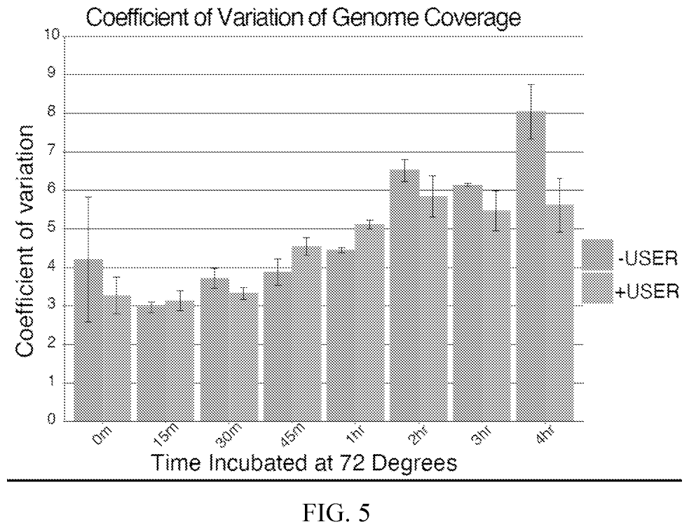

Amplicon libraries of polynucleotides generated from the PTA methods and compositions (terminators, polymerases, etc.) described herein in some instances have increased uniformity. Uniformity, in some instances, is described using a Lorenz curve (e.g., FIG. 5C), or other such method. Such increases in some instances lead to lower sequencing reads needed for the desired coverage of a target nucleic acid molecule (e.g., genomic DNA, RNA, or other target nucleic acid molecule). For example, no more than 50% of a cumulative fraction of polynucleotides comprises sequences of at least 80% of a cumulative fraction of sequences of the target nucleic acid molecule. In some instances, no more than 50% of a cumulative fraction of polynucleotides comprises sequences of at least 60% of a cumulative fraction of sequences of the target nucleic acid molecule. In some instances, no more than 50% of a cumulative fraction of polynucleotides comprises sequences of at least 70% of a cumulative fraction of sequences of the target nucleic acid molecule. In some instances, no more than 50% of a cumulative fraction of polynucleotides comprises sequences of at least 90% of a cumulative fraction of sequences of the target nucleic acid molecule. In some instances, uniformity is described using a Gini index (wherein an index of 0 represents perfect equality of the library and an index of 1 represents perfect inequality). In some instances, amplicon libraries described herein have a Gini index of no more than 0.55, 0.50, 0.45, 0.40, or 0.30. In some instances, amplicon libraries described herein have a Gini index of no more than 0.50. In some instances, amplicon libraries described herein have a Gini index of no more than 0.40. Such uniformity metrics in some instances are dependent on the number of reads obtained.

For example, no more than 100 million, 200 million, 300 million, 400 million, or no more than 500 million reads are obtained. In some instances, the read length is about 50, 75, 100, 125, 150, 175, 200, 225, or about 250 bases in length. In some instances, uniformity metrics are dependent on the depth of coverage of a target nucleic acid. For example, the average depth of coverage is about 10×, 15×, 20×, 25×, or about 30×. In some instances, the average depth of coverage is 10-30×, 20-50×, 5-40×, 20-60×, 5-20×, or 10-20×. In some instances, amplicon libraries described herein have a Gini index of no more than 0.55, wherein about 300 million reads was obtained. In some instances, amplicon libraries described herein have a Gini index of no more than 0.50, wherein about 300 million reads was obtained. In some instances, amplicon libraries described herein have a Gini index of no more than 0.45, wherein about 300 million reads was obtained. In some instances, amplicon libraries described herein have a Gini index of no more than 0.55, wherein no more than 300 million reads was obtained. In some instances, amplicon libraries described herein have a Gini index of no more than 0.50, wherein no more than 300 million reads was obtained. In some instances, amplicon libraries described herein have a Gini index of no more than 0.45, wherein no more than 300 million reads was obtained. In some instances, amplicon libraries described herein have a Gini index of no more than 0.55, wherein the average depth of sequencing coverage is about 15×. In some instances, amplicon libraries described herein have a Gini index of no more than 0.50, wherein the average depth of sequencing coverage is about 15×. In some instances, amplicon libraries described herein have a Gini index of no more than 0.45, wherein the average depth of sequencing coverage is about 15×. In some instances, amplicon libraries described herein have a Gini index of no more than 0.55, wherein the average depth of sequencing coverage is at least 15×. In some instances, amplicon libraries described herein have a Gini index of no more than 0.50, wherein the average depth of sequencing coverage is at least 15×. In some instances, amplicon libraries described herein have a Gini index of no more than 0.45, wherein the average depth of sequencing coverage is at least 15×. In some instances, amplicon libraries described herein have a Gini index of no more than 0.55, wherein the average depth of sequencing coverage is no more than 15×. In some instances, amplicon libraries described herein have a Gini index of no more than 0.50, wherein the average depth of sequencing coverage is no more than 15×. In some instances, amplicon libraries described herein have a Gini index of no more than 0.45, wherein the average depth of sequencing coverage is no more than 15×. Uniform amplicon libraries generated using the methods described herein are in some instances subjected to additional steps, such as adapter ligation and further PCR amplification. In some instances, such additional steps precede a sequencing step.

Primers comprise nucleic acids used for priming the amplification reactions described herein. Such primers in some instances include, without limitation, random deoxynucleotides of any length with or without modifications to make them exonuclease resistant, random ribonucleotides of any length with or without modifications to make them exonuclease resistant, modified nucleic acids such as locked nucleic acids, DNA or RNA primers that are targeted to a specific genomic region, and reactions that are primed with enzymes such as primase. In the case of whole genome PTA, it is preferred that a set of primers having random or partially random nucleotide sequences be used. In a nucleic acid sample of significant complexity, specific nucleic acid sequences present in the sample need not be known and the primers need not be designed to be complementary to any particular sequence. Rather, the complexity of the nucleic acid sample results in a large number of different hybridization target sequences in the sample, which will be complementary to various primers of random or partially random sequence. The complementary portion of primers for use in PTA are in some instances fully randomized, comprise only a portion that is randomized, or be otherwise selectively randomized. The number of random base positions in the complementary portion of primers in some instances, for example, is from 20% to 100% of the total number of nucleotides in the complementary portion of the primers. In some instances, the number of random base positions in the complementary portion of primers is 10% to 90%, 15-95%, 20%-100%, 30%-100%, 50%-100%, 75-100% or 90-95% of the total number of nucleotides in the complementary portion of the primers. In some instances, the number of random base positions in the complementary portion of primers is at least 10%, 20%, 30%, 40%, 50%, 60%, 70%, 80%, or at least 90% of the total number of nucleotides in the complementary portion of the primers. Sets of primers having random or partially random sequences are in some instances synthesized using standard techniques by allowing the addition of any nucleotide at each position to be randomized.

In some instances, sets of primers are composed of primers of similar length and/or hybridization characteristics. In some instances, the term “random primer” refers to a primer which can exhibit four-fold degeneracy at each position. In some instances, the term “random primer” refers to a primer which can exhibit three-fold degeneracy at each position. Random primers used in the methods described herein in some instances comprise a random sequence that is 3, 4, 5, 6, 7, 8, 10, 11, 12, 13, 14, 15, 16, 17, 18, 19, 20, or more bases in length. In some instances, primers comprise random sequences that are 3-20, 5-15, 5-20, 6-12, or 4-10 bases in length. Primers may also comprise non-extendable elements that limit subsequent amplification of amplicons generated thereof. For example, primers with non-extendable elements in some instances comprise terminators. In some instances, primers comprise terminator nucleotides, such as 1, 2, 3, 4, 5, 10, or more than 10 terminator nucleotides. Primers need not be limited to components which are added externally to an amplification reaction. In some instances, primers are generated in-situ through the addition of nucleotides and proteins which promote priming. For example, primase-like enzymes in combination with nucleotides is in some instances used to generate random primers for the methods described herein. Primase-like enzymes in some instances are members of the DnaG or AEP enzyme superfamily. In some instances, a primase-like enzyme is TthPrimPol. In some instances, a primase-like enzyme is T7 gp4 helicase-primase. Such primases are in some instances used with the polymerases or strand displacement factors described herein. In some instances, primases initiate priming with deoxyribonucleotides. In some instances, primases initiate priming with ribonucleotides.

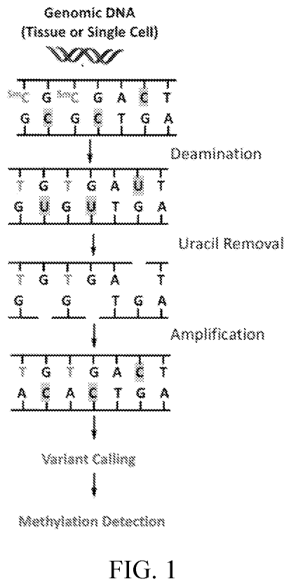

The PTA amplification can be followed by selection for a specific subset of amplicons. Such selections are in some instances dependent on size, affinity, activity, hybridization to probes, or other known selection factor in the art. In some instances, selections precede or follow additional steps described herein, such as adapter ligation and/or library amplification. In some instances, selections are based on size (length) of the amplicons. In some instances, smaller amplicons are selected that are less likely to have undergone exponential amplification, which enriches for products that were derived from the primary template while further converting the amplification from an exponential into a quasi-linear amplification process (FIG. 1A). In some instances, amplicons comprising 50-2000, 25-5000, 40-3000, 50-1000, 200-1000, 300-1000, 400-1000, 400-600, 600-2000, or 800-1000 bases in length are selected. Size selection in some instances occurs with the use of protocols, e.g., utilizing solid-phase reversible immobilization (SPRI) on carboxylated paramagnetic beads to enrich for nucleic acid fragments of specific sizes, or other protocol known by those skilled in the art. Optionally or in combination, selection occurs through preferential ligation and amplification of smaller fragments during PCR while preparing sequencing libraries, as well as a result of the preferential formation of clusters from smaller sequencing library fragments during sequencing (e.g., sequencing by synthesis, nanopore sequencing, or other sequencing method). Other strategies to select for smaller fragments are also consistent with the methods described herein and include, without limitation, isolating nucleic acid fragments of specific sizes after gel electrophoresis, the use of silica columns that bind nucleic acid fragments of specific sizes, and the use of other PCR strategies that more strongly enrich for smaller fragments. Any number of library preparation protocols may be used with the PTA methods described herein. Amplicons generated by PTA are in some instances ligated to adapters (optionally with removal of terminator nucleotides). In some instances, amplicons generated by PTA comprise regions of homology generated from transposase-based fragmentation which are used as priming sites. In some instances, libraries are prepared by fragmenting nucleic acids mechanically or enzymatically. In some instances, libraries are prepared using tagmentation via transposomes. In some instances, libraries are prepared via ligation of adapters, such as Y-adapters, universal adapters, or circular adapters.

The non-complementary portion of a primer used in PTA can include sequences which can be used to further manipulate and/or analyze amplified sequences. An example of such a sequence is a“detection tag”. Detection tags have sequences complementary to detection probes and are detected using their cognate detection probes. There may be one, two, three, four, or more than four detection tags on a primer. There is no fundamental limit to the number of detection tags that can be present on a primer except the size of the primer. In some instances, there is a single detection tag on a primer. In some instances, there are two detection tags on a primer. When there are multiple detection tags, they may have the same sequence or they may have different sequences, with each different sequence complementary to a different detection probe. In some instances, multiple detection tags have the same sequence. In some instances, multiple detection tags have a different sequence.

Another example of a sequence that can be included in the non-complementary portion of a primer is an “address tag” that can encode other details of the amplicons, such as the location in a tissue section. In some instances, a cell barcode comprises an address tag. An address tag has a sequence complementary to an address probe. Address tags become incorporated at the ends of amplified strands. If present, there may be one, or more than one, address tag on a primer. There is no fundamental limit to the number of address tags that can be present on a primer except the size of the primer. When there are multiple address tags, they may have the same sequence or they may have different sequences, with each different sequence complementary to a different address probe. The address tag portion can be any length that supports specific and stable hybridization between the address tag and the address probe. In some instances, nucleic acids from more than one source can incorporate a variable tag sequence. This tag sequence can be up to 100 nucleotides in length, preferably 1 to 10 nucleotides in length, most preferably 4, 5 or 6 nucleotides in length and comprises combinations of nucleotides. In some instances, a tag sequence is 1-20, 2-15, 3-13, 4-12, 5-12, or 1-10 nucleotides in length For example, if six base-pairs are chosen to form the tag and a permutation of four different nucleotides is used, then a total of 4096 nucleic acid anchors (e.g. hairpins), each with a unique 6 base tag can be made.

Primers described herein may be present in solution or immobilized on a solid support. In some instances, primers bearing sample barcodes and/or UMI sequences can be immobilized on a solid support. The solid support can be, for example, one or more beads. In some instances, individual cells are contacted with one or more beads having a unique set of sample barcodes and/or UMI sequences in order to identify the individual cell. In some instances, lysates from individual cells are contacted with one or more beads having a unique set of sample barcodes and/or UMI sequences in order to identify the individual cell lysates. In some instances, extracted nucleic acid from individual cells are contacted with one or more beads having a unique set of sample barcodes and/or UMI sequences in order to identify the extracted nucleic acid from the individual cell. The beads can be manipulated in any suitable manner as is known in the art, for example, using droplet actuators as described herein. The beads may be any suitable size, including for example, microbeads, microparticles, nanobeads and nanoparticles. In some embodiments, beads are magnetically responsive; in other embodiments beads are not significantly magnetically responsive. Non-limiting examples of suitable beads include flow cytometry microbeads, polystyrene microparticles and nanoparticles, functionalized polystyrene microparticles and nanoparticles, coated polystyrene microparticles and nanoparticles, silica microbeads, fluorescent microspheres and nanospheres, functionalized fluorescent microspheres and nanospheres, coated fluorescent microspheres and nanospheres, color dyed microparticles and nanoparticles, magnetic microparticles and nanoparticles, superparamagnetic microparticles and nanoparticles (e.g., DYNABEADS® available from Invitrogen Group, Carlsbad, CA), fluorescent microparticles and nanoparticles, coated magnetic microparticles and nanoparticles, ferromagnetic microparticles and nanoparticles, coated ferromagnetic microparticles and nanoparticles, and those described in U.S. Pat. Appl. Pub. No. US20050260686, US20030132538, US20050118574, 20050277197, 20060159962. Beads may be pre-coupled with an antibody, protein or antigen, DNA/RNA probe or any other molecule with an affinity for a desired target. In some embodiments, primers bearing sample barcodes and/or UMI sequences can be in solution. In certain embodiments, a plurality of droplets can be presented, wherein each droplet in the plurality bears a sample barcode which is unique to a droplet and the UMI which is unique to a molecule such that the UMI are repeated many times within a collection of droplets. In some embodiments, individual cells are contacted with a droplet having a unique set of sample barcodes and/or UMI sequences in order to identify the individual cell. In some embodiments, lysates from individual cells are contacted with a droplet having a unique set of sample barcodes and/or UMI sequences in order to identify the individual cell lysates. In some embodiments, extracted nucleic acid from individual cells are contacted with a droplet having a unique set of sample barcodes and/or UMI sequences in order to identify the extracted nucleic acid from the individual cell.

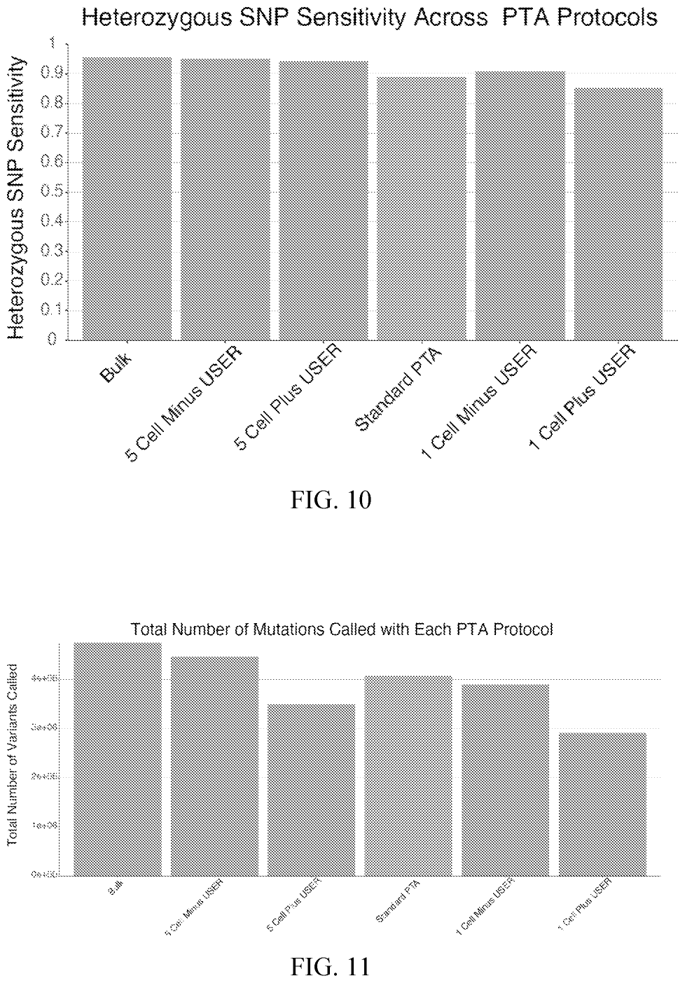

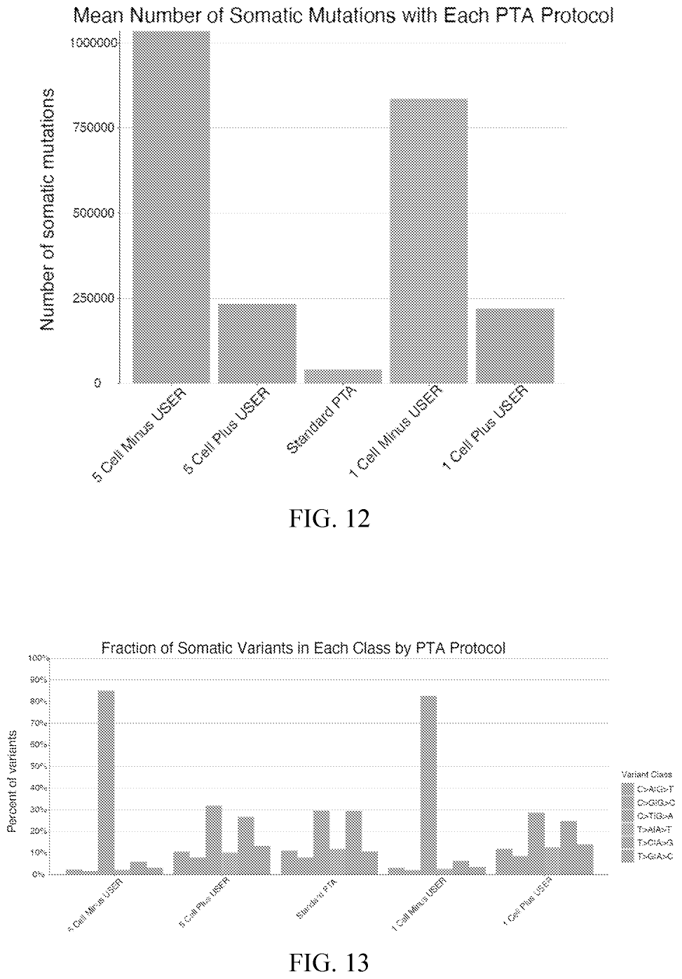

PTA primers may comprise a sequence-specific or random primer, a cell barcode and/or a unique molecular identifier (UMI) (see, e.g., FIGS. 10A (linear primer) and 10B (hairpin primer)). In some instances, a primer comprises a sequence-specific primer. In some instances, a primer comprises a random primer. In some instances, a primer comprises a cell barcode. In some instances, a primer comprises a sample barcode. In some instances, a primer comprises a unique molecular identifier. In some instances, primers comprise two or more cell barcodes. Such barcodes in some instances identify a unique sample source, or unique workflow. Such barcodes or UMIs are in some instances 5, 6, 7, 8, 9, 10, 11, 12, 15, 20, 25, 30, or more than 30 bases in length. Primers in some instances comprise at least 1000, 10,000, 50,000, 100,000, 250,000, 500,000, 106, 107, 108, 109, or at least 1010 unique barcodes or UMIs. In some instances primers comprise at least 8, 16, 96, or 384 unique barcodes or UMIs. In some instances a standard adapter is then ligated onto the amplification products prior to sequencing; after sequencing, reads are first assigned to a specific cell based on the cell barcode. Suitable adapters that may be utilized with the PTA method include, e.g., xGen® Dual Index UMI adapters available from Integrated DNA Technologies (IDT). Reads from each cell is then grouped using the UMI, and reads with the same UMI may be collapsed into a consensus read. The use of a cell barcode allows all cells to be pooled prior to library preparation, as they can later be identified by the cell barcode. The use of the UMI to form a consensus read in some instances corrects for PCR bias, improving the copy number variation (CNV) detection. In addition, sequencing errors may be corrected by requiring that a fixed percentage of reads from the same molecule have the same base change detected at each position. This approach has been utilized to improve CNV detection and correct sequencing errors in bulk samples. In some instances, UMIs are used with the methods described herein, for example, U.S. Pat. No. 8,835,358 discloses the principle of digital counting after attaching a random amplifiable barcode. Schmitt et al and Fan et al. disclose similar methods of correcting sequencing errors. In some instances, a library is generated for sequencing using primers. In some instances, the library comprises fragments of 200-700 bases, 100-1000, 300-800, 300-550, 300-700, or 200-800 bases in length. In some instances, the library comprises fragments of at least 50, 100, 150, 200, 300, 500, 600, 700, 800, or at least 1000 bases in length. In some instances, the library comprises fragments of about 50, 100, 150, 200, 300, 500, 600, 700, 800, or about 1000 bases in length.

The methods described herein may further comprise additional steps, including steps performed on the sample or template. Such samples or templates in some instance are subjected to one or more steps prior to PTA. In some instances, samples comprising cells are subjected to a pre treatment step. For example, cells undergo lysis and proteolysis to increase chromatin accessibility using a combination of freeze-thawing, Triton X-100, Tween 20, and Proteinase K. Other lysis strategies are also be suitable for practicing the methods described herein. Such strategies include, without limitation, lysis using other combinations of detergent and/or lysozyme and/or protease treatment and/or physical disruption of cells such as sonication and/or alkaline lysis and/or hypotonic lysis. In some instances, the primary template or target molecule(s) is subjected to a pre-treatment step. In some instances, the primary template (or target) is denatured using sodium hydroxide, followed by neutralization of the solution. Other denaturing strategies may also be suitable for practicing the methods described herein. Such strategies may include, without limitation, combinations of alkaline lysis with other basic solutions, increasing the temperature of the sample and/or altering the salt concentration in the sample, addition of additives such as solvents or oils, other modification, or any combination thereof. In some instances, additional steps include sorting, filtering, or isolating samples, templates, or amplicons by size. In some instances, cells are lysed with mechanical (e.g., high pressure homogenizer, bead milling) or non-mechanical (physical, chemical, or biological). In some instances, physical lysis methods comprise heating, osmotic shock, and/or cavitation. In some instances, chemical lysis comprises alkali and/or detergents. In some instances, biological lysis comprises use of enzymes. Combinations of lysis methods are also compatible with the methods described herein. Non-limited examples of lysis enzymes include recombinant lysozyme, serine proteases, and bacterial lysins. In some instances, lysis with enzymes comprises use of lysozyme, lysostaphin, zymolase, cellulose, protease or glycanase. For example, after amplification with the methods described herein, amplicon libraries are enriched for amplicons having a desired length. In some instances, amplicon libraries are enriched for amplicons having a length of 50-2000, 25-1000, 50-1000, 75-2000, 100-3000, 150-500, 75-250, 170-500, 100-500, or 75-2000 bases. In some instances, amplicon libraries are enriched for amplicons having a length no more than 75, 100, 150, 200, 500, 750, 1000, 2000, 5000, or no more than 10,000 bases. In some instances, amplicon libraries are enriched for amplicons having a length of at least 25, 50, 75, 100, 150, 200, 500, 750, 1000, or at least 2000 bases.

Methods and compositions described herein may comprise buffers or other formulations. Such buffers are in some instances used for PTA, RT, or other method described herein. Such buffers in some instances comprise surfactants/detergent or denaturing agents (Tween-20, DMSO, DMF, pegylated polymers comprising a hydrophobic group, or other surfactant), salts (potassium or sodium phosphate (monobasic or dibasic), sodium chloride, potassium chloride, TrisHCl, magnesium chloride or sulfate, Ammonium salts such as phosphate, nitrate, or sulfate, EDTA), reducing agents (DTT, THP, DTE, beta-mercaptoethanol, TCEP, or other reducing agent) or other components (glycerol, hydrophilic polymers such as PEG). In some instances, buffers are used in conjunction with components such as polymerases, strand displacement factors, terminators, or other reaction component described herein. In some instances, buffers are used in conjunction with components such as polymerases, strand displacement factors, terminators, or other reaction component described herein. Buffers may comprise one or more crowding agents. In some instances, crowding reagents include polymers. In some instances, crowding reagents comprise polymers such as polyols. In some instances, crowding reagents comprise polyethylene glycol polymers (PEG). In some instances, crowding reagents comprise polysaccharides. Without limitation, examples of crowding reagents include ficoll (e.g., ficoll PM 400, ficoll PM 70, or other molecular weight ficoll), PEG (e.g., PEG1000, PEG 2000, PEG4000, PEG6000, PEG8000, or other molecular weight PEG), dextran (dextran 6, dextran 10, dextran 40, dextran 70, dextran 6000, dextran 138 k, or other molecular weight dextran).

The nucleic acid molecules amplified according to the methods described herein may be sequenced and analyzed using methods known to those of skill in the art. Non-limiting examples of the sequencing methods which in some instances are used include, e.g., sequencing by hybridization (SBH), sequencing by ligation (SBL) (Shendure et al. (2005) Science 309: 1728), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (Int. Pat. Appl. Pub. No. WO2006/073504), multiplex sequencing (U.S. Pat. Appl. Pub. No. US2008/0269068; Porreca et al., 2007, Nat. Methods 4:931), polymerized colony (POLONY) sequencing (U.S. Pat. Nos. 6,432,360, 6,485,944 and 6,511,803, and Int. Pat. Appl. Pub. No. WO2005/082098), nanogrid rolling circle sequencing (ROLONY) (U.S. Pat. No. 9,624,538), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout), high-throughput sequencing methods such as, e.g., methods using Roche 454, Illumina Solexa, AB-SOLiD, Helicos, Polonator platforms and the like, and light-based sequencing technologies (Landegren et al. (1998) Genome Res. 8:769-76; Kwok (2000) Pharmacogenomics 1:95-100; and Shi (2001) Clin. Chem. 47: 164-172). In some instances, the amplified nucleic acid molecules are shotgun sequenced. Sequencing of the sequencing library is in some instances performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis (array/colony-based or nanoball based).

Sequencing libraries generated using the methods described herein (e.g., PTA or RNAseq) may be sequenced to obtain a desired number of sequencing reads. In some instances, libraries are generated from a single cell or sample comprising a single cell (alone or part of a multiomics workflow). In some instances, libraries are sequenced to obtain at least 0.1, 0.2, 0.4, 0.5, 0.7, 0.8, 0.9, 1, 1.1, 1.2, 1.5, 2, 5, or at least 10 million reads. In some instances, libraries are sequenced to obtain no more than 0.1, 0.2, 0.4, 0.5, 0.7, 0.8, 0.9, 1, 1.1, 1.2, 1.5, 2, 5, or no more than 10 million reads. In some instances, libraries are sequenced to obtain about 0.1, 0.2, 0.4, 0.5, 0.7, 0.8, 0.9, 1, 1.1, 1.2, 1.5, 2, 5, or about 10 million reads. In some instances, libraries are sequenced to obtain 0.1-10, 0.1-5, 0.1-1, 0.2-1, 0.3-1.5, 0.5-1, 1-5, or 0.5-5 million reads per sample. In some instances, the number of reads is dependent on the size of the genome. In some in instances samples comprising bacterial genomes are sequenced to obtain 0.5-1 million reads. In some instances, libraries are sequenced to obtain at least 2, 4, 10, 20, 50, 100, 200, 300, 500, 700, or at least 900 million reads. In some instances, libraries are sequenced to obtain no more than 2, 4, 10, 20, 50, 100, 200, 300, 500, 700, or no more than 900 million reads. In some instances, libraries are sequenced to obtain about 2, 4, 10, 20, 50, 100, 200, 300, 500, 700, or about 900 million reads. In some in instances samples comprising mammalian genomes are sequenced to obtain 500-600 million reads. In some instances, the type of sequencing library (cDNA libraries or genomic libraries) are identified during sequencing. In some instances, cDNA libraries and genomic libraries are identified during sequencing with unique barcodes.

The term “cycle” when used in reference to a polymerase-mediated amplification reaction is used herein to describe steps of dissociation of at least a portion of a double stranded nucleic acid (e.g., a template from an amplicon, or a double stranded template, denaturation). hybridization of at least a portion of a primer to a template (annealing), and extension of the primer to generate an amplicon. In some instances, the temperature remains constant during a cycle of amplification (e.g., an isothermal reaction). In some instances, the number of cycles is directly correlated with the number of amplicons produced. In some instances, the number of cycles for an isothermal reaction is controlled by the amount of time the reaction is allowed to proceed.

Finally, provided herein are kits for performing the claimed methods. The kits can include reagents necessary for carrying out deamination of the cytosines and modified cytosines and/or reagents for carrying out primary directed-template amplification. Thus, the kits can include one or more enzymes, buffers, e.g., lysis buffers, primers, and combinations thereof.

It is understood that the examples and embodiments described herein are for illustrative purposes only and that various modifications or changes in light thereof will be suggested to persons skilled in the art and are to be included within the spirit and purview of this application and scope of the appended claims.

All publications, patents, and patent applications cited herein are hereby incorporated by reference with respect to the material for which they are expressly cited.

### Example

### Example 1. Genome-Wide Single-Cell Cytosine Methylation and Variant Detection Using Methyl-Primary Template-Directed Amplification

### Methods

Cell Culture and Sorting. Lymphoblastoid cell line GM12878 (Coriell Institute, Camden, NJ, USA) was maintained in RPMI media, which was supplemented with 15% FBS, 2 mM L-glutamine, 100 units/mL of penicillin, 100 μg/mL of streptomycin, and 0.25 μg/mL of Amphotericin B (Thermo Fisher). Cells underwent washing and sorting as previously described, with the exception that they were sorted into 3 ul of ResolveDNA cell buffer (BioSkryb Genomics). Calcein AM-positive, PI-negative cells were sorted using a Sony SH800 sorter where empty wells and five cell controls were also included. Cells were stored at −80° C. for for at least 8 hours until they were ready for analysis.

Methyl-Primary Template Amplification (PTA). The cells were placed in at room temperature for 20 minutes prior to analysis. To prevent evaporation, increased volumes of the ResolveDNA kit (BioSkryb Genomics) were utilized. Specifically, 4.5 ul of MS buffer was added and the cells were incubated at 72 degrees for 30 minutes. 4.5 ul of SN1 was added, followed by 4.33 ul of SDX, 8.66 ul of Reaction PreMix, and 1 ul of USER enzyme (New England Biolabs). The reaction was then incubated at 37 degrees for 30 minutes. We then added 1.16 ul of SEZ1 and 1.7 ul of SEZ2. The reactions were then incubated at 30 degrees for 10 hours and the reaction was heat inactivated at 65 degrees for 10 minutes. Samples then underwent cleanup and Nextera Flex (Illumina) library preparation per manufacturer's instructions. The final libraries were quantified using the Qubit dsDNA BR Kit (Thermo Fisher) and the D1000 ScreenTape Assay with the TapeStation 2200 before sequencing on the NovaSeq 6000 (Illumina). For the time course experiment, reactions were performed in Polymerase Chain Reaction (PCR) tubes and removed at the stated incubated times.

Methyl-PTA Computational Analyses. Raw fastq files were trimmed using Trimmomatic, aligned to GRCh38 using BWA (0.7.12), and processed using GATK 4.0.1 best practices without deviation from the recommended parameters. Coverage, alignment, and other metrics were obtained from the final bam using Picard AlignmentMetricsAummary and CollectWgsMetrics. Genotyping was performed with GATK HaplotypeCaller using—tranche 99.0. We then compared each variant call to the platinum genome in the bottle reference for GM12878 to identify putative 5mC variants. To identify sites of methylation of regulatory regions, the vcf file containing variants not found in the platinum reference genome that were present in enhancers, CpG Islands, or promoters were identified using bedtools. The rate of putative cytosine methylation within each regulatory region was then computed and converted to a digital present absent methylation call. In sites that were determined to be methylated, the allele frequency of those variants and surrounding germline variants were used to predict if one or both alleles were methylated.

### Results

### Methyl-PTA Time Course Experiment

Single cells were isolated in 96 well plates using flow-activated cell sorting, followed by heating to 72 degrees under alkaline conditions, reaction neutralization, uracil removal, and PTA, as outlined in FIG. 1. For the first experiment, lysed single NA12878 cells were removed after different times of exposure to alkaline conditions at 72 degrees and one set of samples underwent uracil removal with USER enzyme while paired cells underwent mock uracil removal. The samples then underwent library preparation with the Nextera Flex system and sequencing on an Illumina Nextseq 2000 where about 10 million reads per sample were obtained.

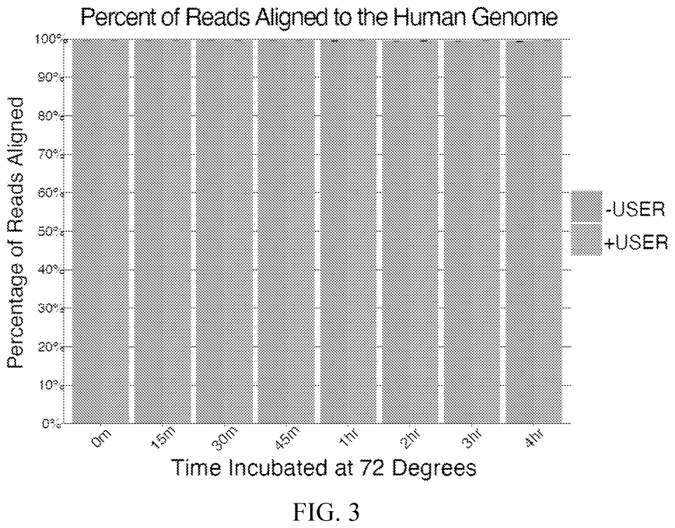

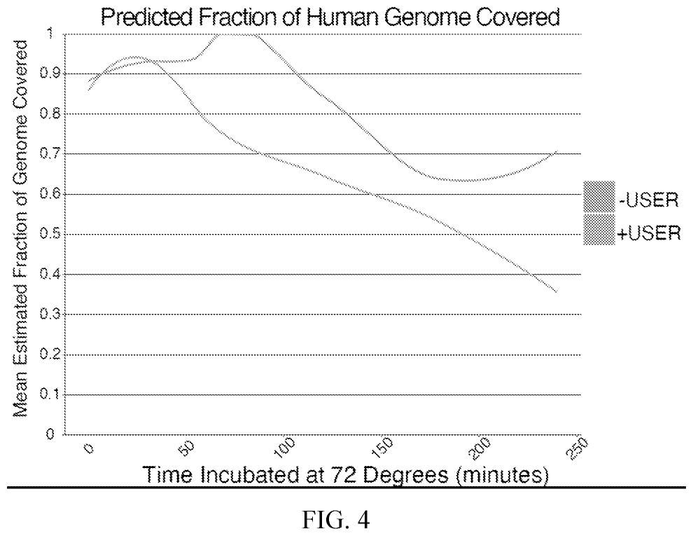

With this approach, the PTA yield decreased over time, suggesting degradation of the primary template with exposure to the alkaline conditions at elevated temperature. In addition, it was noted that the USER enzyme further decreased yield, suggested that uracil removal resulted in additional template degradation (FIG. 2). As seen in FIG. 3, almost all PTA amplicons aligned to the human genome, suggesting there was mostly direct copying of the DNA, and that the reaction was not producing nonspecific byproducts. When using the library complexity to estimate genome coverage using the program PreSeq (http://smithlabresearch.org/software/preseq/), it was estimated that greater than ninety percent of the genome was covered at early timepoints while extended exposure to heat under alkaline conditions led to progressive loss in predicted coverage over time, consistent with the decreased yield in FIG. 2. Further, the uracil removal step appears to lead to increased loss of genome coverage relative to the mock treated sample, which is likely due to small fragments being produced with uracil removal (FIG. 4). Consistent with this finding, the coefficient of variation of coverage increased with time incubated at 72 degrees due to loss of genome coverage, although there was not a clear difference between cells that underwent uracil removal and those that did not (FIG. 5).

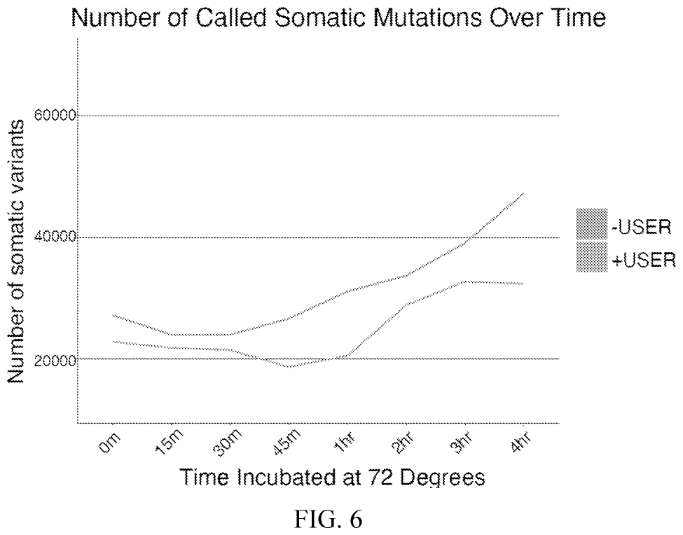

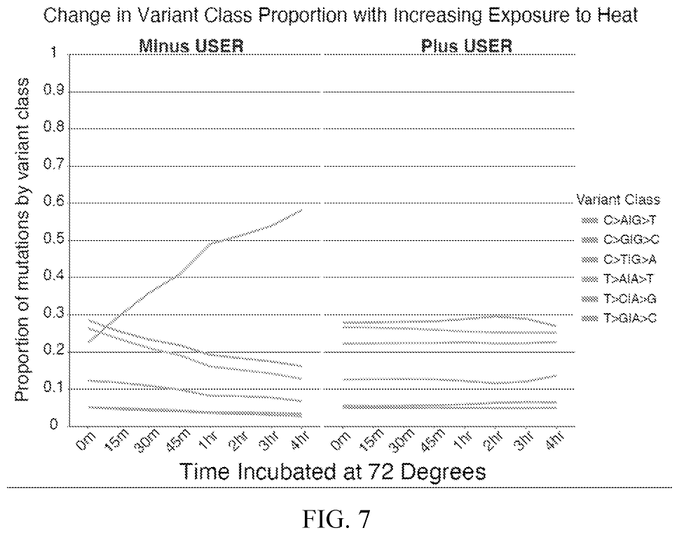

To determine the number and type of mutations that were created as a result of exposure to our deamination conditions, the low pass sequencing data was used to call genomic variants in each cell that were not found in the corresponding bulk samples. As seen in FIG. 6, an increasing mean number of somatic variants over time that decrease in magnitude with uracil removal was observed. As expected, the majority of excess variants seen in the mock USER treatment group were cytosine deamination events (cytosine to thymine or guanine to adenine on the opposite strand, FIG. 7). It is also noted that the proportion of cytosine deamination events does not increase in time with USER treatment, suggesting that most of the previously unmethylated cytosines that had been converted to uracil were removed by the USER enzyme.

### Deep Sequencing of Methyl-PTA Cells

The initial experiment demonstrated that increasing cytosine deamination that is proportional to the time DNA is exposed to alkaline conditions at 72 degrees can be produced. Further, the unmethylated cytosines that underwent deamination into uracil can be efficiently removed. Deep sequencing measurements of the genome coverage was then provided, as well as mutation types and rates detected in each cell. To accomplish this, the same protocol with 30 minutes of deamination incubation with samples with and without the USER enzyme mix for uracil removal was used.

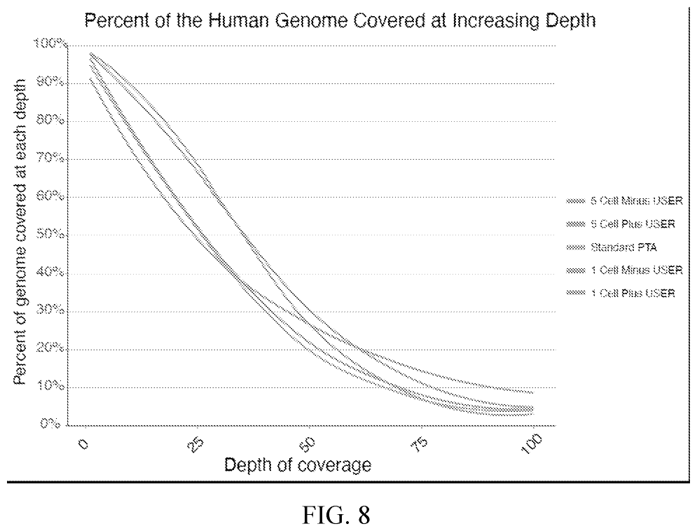

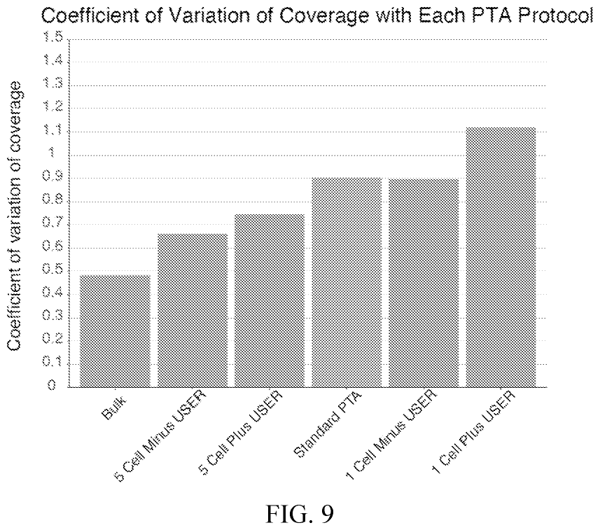

As seen in FIG. 8, all samples, including the USER treated single cells, achieved greater than 90% genome coverage at 1×, and greater than 80% of the genome at 10×. Consistent with the time course experiment, it was also observed the coefficient of variation of coverage increased with USER treatment (FIG. 8). From these data, it was also estimated the heterozygous single nucleotide polymorphism detection sensitivity, which remained 0.85 with the deaminated cells that underwent USER treatment.

Genomic variants were called across all the samples to identify variants detected in the methyl-PTA samples but not in the standard PTA or bulk samples. As expected, the highest number of high-quality variants were called in the bulk sample. In the methyl-PTA samples with USER treatment, around 3 million SNPs were still accurately called. The variants not detected in the bulk sample where it was found that the 30-minute deamination incubation resulted in the creation of about 800,000 somatic marks per cell were examined, most of which were removed with USER treatment. Finally, as identified in the time course experiment, most of the somatic variants in the methyl-PTA protocol were cytosine deamination events, and most of those events were efficiently removed with USER treatment with the leftover cytosine to thymine somatic variants marking 5mC sites.

Taken together, these experiments demonstrate marks can be produced at both methylated and unmethylated cytosines, which are proportional to the time the template DNA is exposed to heat and alkaline conditions. It was also demonstrated that the unmethylated cytosine that have been converted to uracil can be removed, leaving marks at 5mC sites. This results in an easily executed protocol that can identify both genetic variants and marked sites of cytosine methylation in the same cells to begin to more deeply understand how somatic variants interact with epigenetic states in a given cell.

### Example 2. Single-Cell Methyl-Primary Template-Directed Amplification Optimization Experiments (scMethyl-PTA)

Reactions were assembled in a pre-PCR workstation that was decontaminated with ultraviolet light for 30 min before each experiment. scMethyl-PTA was carried out by first lysing the cell and denaturing the DNA in the presence of additional DTT and/or MgCl₂. The cells were then vortexed and briefly centrifuged before incubating at 56 or 60° C. for four hours. Next, lysis was stopped and denaturation commenced by adding BioSkryb Stop solution, vortexing, spinning, and proceeding to enzymatic uracil removal using the USER enzyme (New England Biolabs) for 30 min at 37° C. This was followed by PTA for 10 hours at 30° C. after which the amplification was terminated by heating to 65° C. for 3 min (ResolveDNA Kit, BioSkryb Genomics, Durham, N.C.). After the scMethyl-PTA, the deaminated and amplified DNA was purified using AMPure XP magnetic beads (Beckman Coulter, Brea, CA) at a 2:1 ratio of beads to sample volume, and the yields were measured using the Qubit double stranded DNA (dsDNA) HS Assay Kit with a Qubit 3.0 fluorometer according to the manufacturer's instructions (Thermo Fisher).

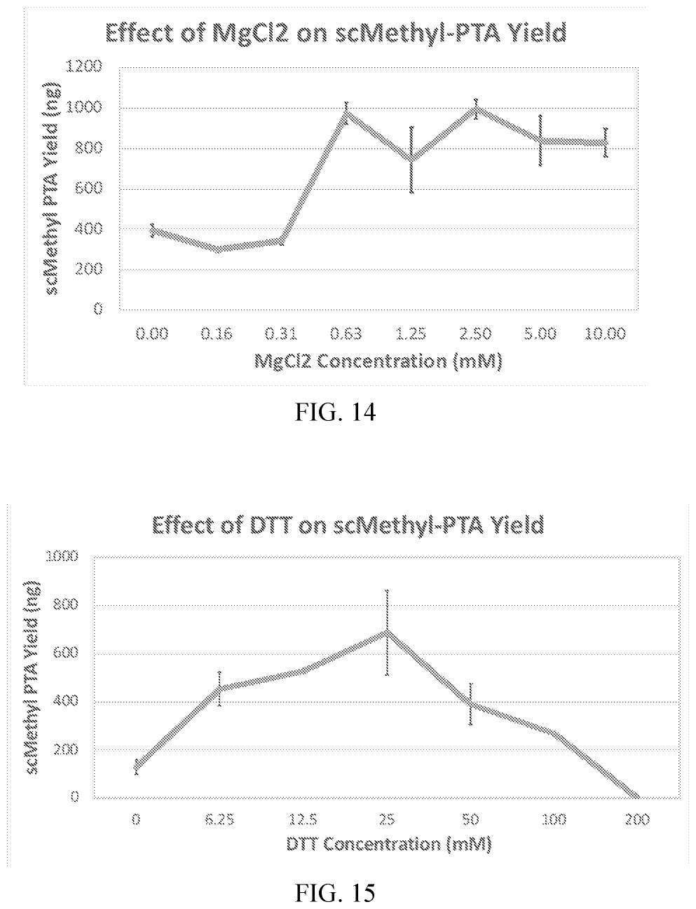

FIG. 14 shows the effects of MgCL₂ on single-cell-Methyl-PTA. Specifically, FIG. 14 shows MgCl₂ increases the yield from single-cell-Methyl-PTA. Single GM12878 cells sorted into LoBind PCR tubes containing 3 μL of BioSkryb cell buffer were lysed and heat-induced cytosine-deaminated at 60° C. for 4 hours in the presence of various concentrations of MgCl₂ followed by uracil removal with USER and PTA. The line graph indicates the median DNA yield from three independent scMethyl-PTA experiments, and the corresponding standard error.

FIG. 15 shows the effect of dithiothreitol (DTT) on single-cell-Methyl-PTA. Specifically, FIG. 15 shows DTT increases the yield from single-cell-Methyl-PTA. Single GM12878 cells sorted into LoBind PCR tubes containing 3 μL of BioSkryb cell buffer were lysed and heat-induced cytosine-deaminated at 60° C. for 4 hours in the presence of various concentrations of DTT followed by uracil removal and PTA. The line graph indicates the median DNA yield from three independent scMethyl-PTA experiments, and the corresponding standard error.

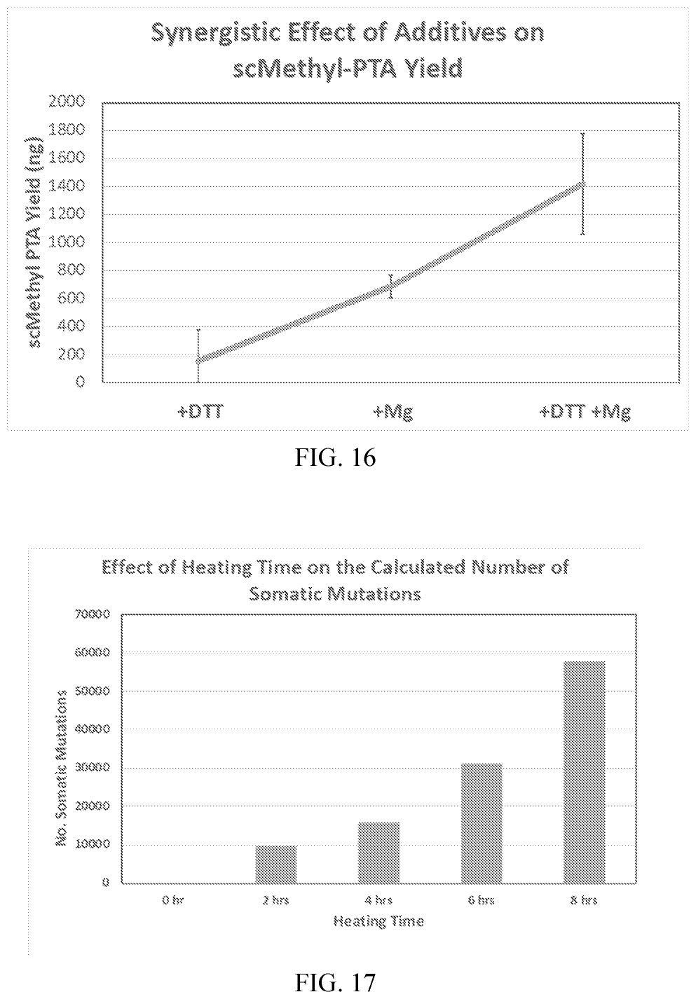

FIG. 16 shows MgCl₂ and DTT additives have a synergistic effect on the yield from single-cell-Methyl-PTA. Single GM12878 cells sorted into LoBind PCR tubes containing 3 μL of BioSkryb cell buffer were lysed and heat-induced Cytosine-deaminated at 56° C. for 4 hours in the presence of 0.63 mM MgCl₂ and/or 25 mM DTT followed by uracil removal and PTA. The line graph indicates the median DNA yield from three independent scMethyl-PTA experiments, and the corresponding standard error.

### Example 3. scMethyl-PTA Time Course Experiment

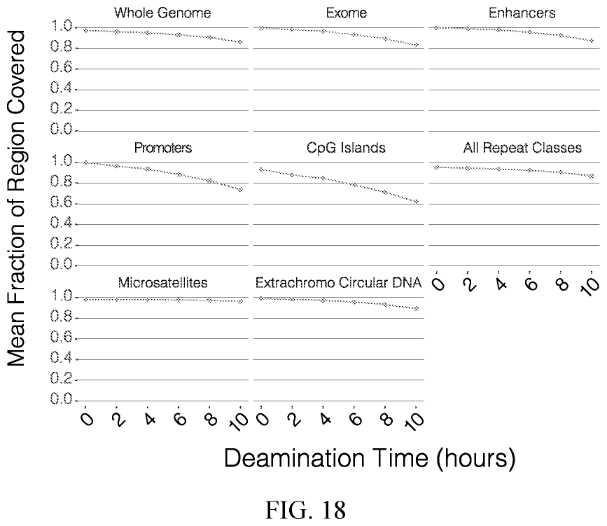

Reactions were assembled in a pre-PCR workstation that was decontaminated with ultraviolet light for 30 minutes before each experiment. scMethyl-PTA was carried out by first lysing the cell and denaturing the DNA in the presence of DTT and MgCl₂. The cells were then vortexed and briefly centrifuged before incubating at 56° C. for various amounts of time. Next, lysis was stopped and denaturation commenced by adding BioSkryb Stop solution, vortexing, spinning, and proceeding to uracil removal using USER enzyme for 30 minutes at 37° C. This was followed by running PTA for 10 hours at 30° C. after which the amplification was terminated by heating to 65° C. for 3 minutes. After the scMethyl-PTA the C-deaminated and amplified DNA was purified using AMPure XP magnetic beads (Beckman Coulter, Brea, CA) at a 2:1 ratio of beads to sample volume, and the resulting amplified DNA was quantified using the Qubit double stranded DNA (dsDNA) HS Assay Kit with a Qubit 3.0 fluorometer. Sequencing libraries were then prepared using Nextera Flex DNA library prep kit, and then underwent deep whole genome sequencing on an Illumina NovaSeq sequencer. The subsequent fastq files were aligned, deduplicated, and underwent variant quality score recalibration using the Sentieon germline calling pipeline with standard parameters. This was followed by somatic variant calling of the bam files and genome wide somatic mutation number calculation using SCAN2. The results are shown in FIGS. 17 and 18.

FIG. 17 shows longer heat-induced deamination step of single-cell-Methyl-PTA results in an increased number of somatic mutations. Single GM12878 cells sorted into LoBind PCR tubes containing 3 μL of BioSkryb cell buffer were lysed and heat-induced Cytosine-deaminated at 56° C. for various amounts of time in the presence of 0.63 mM MgCl₂ and/or 25 mM DTT followed by uracil removal with USER enzyme and PTA. Sequencing libraries were prepared using Nextera Flex DNA library prep kit, and then underwent 30× whole genome sequencing followed by variant calling and whole genome somatic mutation number estimates using SCAN2. The graph indicates the calculated number of somatic mutations.

FIG. 18 shows longer heat-induced deamination step of single-cell-Methyl-PTA results in selective depletion of promoters and CpG islands. Single GM12878 cells sorted into LoBind PCR tubes containing 3 μL of BioSkryb cell buffer were lysed and heat-induced Cytosine-deaminated at 56° C. for various amounts of time in the presence of 0.63 mM MgCl₂ and/or 25 mM DTT followed by uracil removal with USER enzyme and PTA. As described above, sequencing libraries were prepared using Nextera Flex DNA library prep kit, and then underwent 30× whole genome sequencing followed by alignment using Sentieon and calculation of the fraction of specific genomic regions covered using a bed files downloaded from the University of California Santa Cruz Table Browser with Mosdepth. As expected, depletion of regions with high rates of cytosine was observed including CpG Islands that have lower rates of 5-methyl-cytosine at CpG sites than other regions of the genome.

1. A method of identifying modified cytosines in genomic DNA in a biological sample, the method comprising the steps of:
(a) isolating, from the biological sample, nucleic acids comprising genomic DNA comprising cytosines and modified cytosines;
(b) contacting the isolated genomic DNA under conditions resulting in deamination of the genomic DNA thereby converting at least some of the cytosines in the genomic DNA to uracil and at least some of the modified cytosines to thymine;
(c) contacting the deaminated, isolated the genomic DNA with an enzyme to remove uracil from the genomic DNA;
(d) amplifying the genomic DNA lacking uracil using primary-directed template amplification to produce a genomic DNA library; and
(e) sequencing the genomic DNA library, wherein the sequencing identifies the modified cytosines in the genomic DNA of the single cell.

2. The method of claim 1, wherein the modified cytosines are 5′-methylcytosines or 5′ hydroxymethylcytosines.

3. The method of claim 2, wherein identifying the methylated cytosines in the genomic DNA comprises identifying CpG islands in the genomic DNA.

4. The method of claim 2, wherein deamination comprises contacting the isolated genomic DNA with an enzyme that converts cytosine to uracil and 5′-methylcytosine to thymine.

5. The method of claim 4, wherein the enzyme is APOBEC1 (A1), Activation Induced Deaminase (AID), APOBEC2 (A2), APOBEC3A-H (A3A-H), APOBEC4 (A4) or combinations thereof.

6. (canceled)

7. The method of claim 2, wherein deamination comprises contacting the isolated genomic DNA with an enzyme converting cytosine to uracil and 5′-hydroxymethylcytosine to thymine.

8. The method of claim 1, wherein the contacting of step (b) comprises incubating the genomic DNA at a temperature between 50° C. and 100° C. or between 50° C. and 65° C.

9. (canceled)

10. The method of claim 1, wherein the contacting of step (b) occurs over a period of time of between four (4) to twenty four (24) hours.

11. (canceled)

12. The method of claim 1, wherein the contacting of step (b) comprises contacting the biological sample with a lysis buffer with a pH of between 9 to 14.

13. (canceled)

14. The method of claim 1, wherein the contacting of step (c) comprises contacting the deaminated, isolated genomic DNA with an enzymatic buffer comprising the enzyme.

15. The method of claim 14, wherein the enzyme is uracil DNA glycosylase.

16. The method of claim 15, wherein the enzymatic buffer further comprises DNA glycosylase-lyase Endonuclease VIII.

17. The method of claim 1, wherein the contacting of step (c) occurs over a period of time of between 15 to 60 minutes.

18. (canceled)

19. The method of claim 1, wherein the contacting of step (c) comprises incubating the genomic DNA at a temperature from 30° C. to 45° C.

20-26. (canceled)

27. The method of claim 26, wherein the biological sample is a low input sample selected from the group consisting of a forensic sample, ancient genomic fragments, and unculturable microbes.

28. The method of claim 26, wherein the biological sample is a single cell.

29-40. (canceled)

41. The method of claim 1, wherein the primary-directed template amplification comprises:
(i) contacting the genomic DNA with at least one amplification primer, at least one nucleic acid polymerase, and a mixture of nucleotides, wherein the mixture of nucleotides comprises at least one terminator nucleotide which terminates nucleic acid replication by the polymerase;
(ii) amplifying at least some of the genomic DNA to generate a plurality of terminated amplification products, wherein the replication proceeds by strand displacement replication; and
(iii) ligating the molecules obtained in step (ii) to adaptors, thereby generating a genomic DNA library.

42. The method of claim 41, wherein the method further comprises removing at least one terminator nucleotide from the terminated amplification products.

43-44. (canceled)

45. The method of claim 41, wherein at least some of the amplification products comprise a cell barcode or a sample barcode.

46. The method of claim 1, wherein the method is carried out in the presence of magnesium, DTT or both.

47. (canceled)
